(19) United States
(12) Patent Application Publication
(10) Pub. No.: US 2014/0271542 A1
(43) Pub. Date: Sep. 18, 2014

(54) GENETIC MARKER FOR PREDICTING PROGNOSIS IN PATIENTS INFECTED WITH HEPATITIS C VIRUS

(71) Applicant: THE UNITED STATES OF AMERICA as represented by THE SECRETARY, DEPARTMENT OF HEALTH AND HUMAN SERVICE, Bethesda, MD (US)

(72) Inventors: Liudmila Prokunina, Rockville, MD (US); Thomas O'Brien, Potomac, MD (US); Brian Muchmore, Potomac, MD (US); Raymond Donnelly, Damascus, MD (US)

(21) Appl. No.: 14/349,395

(22) PCT Filed: Oct. 5, 2012
(86) PCT No.: PCT/US2012/059048

§ 371 Date: Apr. 3, 2014

Related U.S. Application Data
(60) Provisional application No. 61/543,620, filed on Oct. 5, 2011.

(51) Int. Cl. C12Q 1/70

(52) U.S. Cl. CPC C12Q 1/707
USPC 424/85.4; 435/5; 514/43

(57) ABSTRACT

The present invention provides methods and materials for predicting the clinical outcome of a hepatitis C virus (HCV) infection. In particular, the invention relates to methods and materials for predicating the ability of an individual to spontaneously clear a hepatitis C virus infection. It also relates to methods and materials useful for predicting the clinical response of an individual suffering from HCV infection to administration of a therapeutic treatment. Also provided are kits for predicting the ability of an individual to spontaneously clear an HCV infection, or spontaneously clear such an infection. The methods and rated kits disclosed herein can also be used to develop a treatment plan for an individual infected with HCV, or who is at risk for developing such an infection.

### FIELD OF THE INVENTION

The present invention relates to methods of determining the clinical outcome of an HCV infection by determining the forms of a specific, novel polymorphism present in the individual.

### BACKGROUND

Hepatitis C virus (HCV) is a single-stranded RNA virus in the Flaviviridae family of viruses. It is estimated that there are approximately 170 million persons worldwide and at least 4 million in the United States who have been infected with HCV (Thomas D L, Astemborski J, Rai R M, Anania F A, Schaeffer M, Galai N, Nolt K, Nelson K E, Strathdee S A, Johnson L, Laeyendecker O, Boitnott J, Wilson L E, Vlahov D., The Natural History of Hepatitis C Virus Infection. JAMA 2000; 284 (4): 450-456). Thus, infection with HCV represents a significant, worldwide health problem.

In most people, acute infection with HCV generally results in mild symptoms such as fatigue, decreased appetite, and flu-like symptoms. By convention, acute hepatitis refers to the presence of clinical signs or symptoms of hepatitis for a period of 6 months or fewer after the presumed time of exposure (Blackard J T, Shata M T, Shire N J, Sherman K E., Acute Hepatitis C Virus Infection: A Chronic Problem, Hepatology 2008; 47(1):321-331). In some instances, however, the newly infected individual remains asymptomatic. While some individuals can spontaneously clear the virus, approximately 85% of people infected with HCV will develop chronic hepatitis C, which is defined as persistent viremia occurring at least 6 months after initial exposure (Blackard J T, Shata M T, Shire N J, Sherman K E., Acute Hepatitis C Virus Infection: A Chronic Problem., Hepatology 2008; 47(1):321-331). Chronic infection with HCV is a leading cause of liver cancer and end-stage liver disease. It is also the most common reason for liver transplantation in the U.S. Currently, the standard treatment for HCV infections is pegylated interferon-α (IFN-α) combined with ribavirin. Successful treatment resolves chronic HCV infection, thereby markedly reducing HCV related morbidity and mortality, but the pegylated IFN-α/ribavirin regimen is effective in less than 45% of patients, is expensive and has many adverse effects. More recently, a triple therapy comprising pegylated-IFN-α, ribavirin, and an HCV protease inhibitor has been recommended. Although this new regimen should be more efficacious than treatment with pegylated-interferon-α/ribavirin, a sizeable proportion of patients may fail to respond and patients treated with this regimen will experience the adverse effects seen with pegylated-IFN-α/ribavirin therapy. Thus, a method of identifying patients who are unlikely to respond to treatment with interferon-based therapies is urgently desired so that these patients can be spared the expense and adverse effects associated with futile treatment.

Increasing evidence suggests that host genetic factors influence both the natural course of chronic HCV infection and response to therapy (Lauer G M, Walker B D. Hepatitis C virus infection. N Engl J Med 2001 Jul. 5; 345(1):41-52; Manns M P, McHutchison J G, Gordon S C, Rustgi V K, Shiffman M, Reindollar R, et al. Peginterferon alfa-2b plus ribavirin compared with interferon alfa-2b plus ribavirin for initial treatment of chronic hepatitis C: a randomised trial. Lancet 2001 Sep. 22; 358(9286):958-965; Fried M W, Shiffman M L, Reddy K R, Smith C, Marinos G, Goncales F L, Jr., et al. Peginterferon alfa-2a plus ribavirin for chronic hepatitis C virus infection. N Engl J Med 2002 Sep. 26; 347(13):975-982; Kau A, Vermehren J, Sarrazin C. Treatment predictors of a sustained virologic response in hepatitis B and C. J Hepatol 2008 October; 49(4):634-651). For example, in two cohorts of pregnant women infected under similar conditions from immunoglobulin preparations contaminated with a single strain of HCV, half spontaneously cleared the infection and half progressed to chronic hepatitis C (Grakoui A, Shoukry N H, Woollard D J, Han J H, Hanson H L, Ghrayeb J, et al. HCV persistence and immune evasion in the absence of memory T cell help. Science 2003 Oct. 24; 302(5645):659-662; Knapp S, Yee L J, Frodsham A J, Hennig B J, Hellier S, Zhang L, et al. Polymorphisms in interferon-induced genes and the outcome of hepatitis C virus infection: roles of MxA, OAS-1 and PKR. Genes Immun 2003 September; 4(6):411-419). Among chronically infected patients, response to treatment differs, even between cases with similar HCV-RNA levels and identical genotypes (Thio C L. Host genetic factors and antiviral immune responses to hepatitis C virus. Clin Liver Dis 2008 August; 12(3):713-26, xi; Yee L J. Host genetic determinants in hepatitis C virus infection. Genes Immun 2004 June; 5(4):237-245; Muller R. The natural history of hepatitis C: clinical experiences. J Hepatol 1996; 24(2 Suppl):52-54). The response rates are strongly associated with ethnicity (Knapp S, Hennig B J, Frodsham A J, Zhang L, Hellier S, Wright M, et al. Interleukin-10 promoter polymorphisms and the outcome of hepatitis C virus infection. Immunogenetics 2003 September; 55(6):362-369). Previous reports revealed the influence of genetic polymorphisms of human leukocyte antigens (HLA) (Lauer G M, Walker B D. Hepatitis C virus infection. N Engl J Med 2001 Jul. 5; 345(1):41-52; Kau A, Vermehren J, Sarrazin C. Treatment predictors of a sustained virologic response in hepatitis B and C. J Hepatol 2008 October; 49(4):634-651), killer immunoglobulin-like receptors (KIRs) (Wietzke-Brann P, Maouzi A B, Manhardt L B, Bickeboller H, Ramadori G, Mihm S. Interferon regulatory factor-1 promoter polymorphism and the outcome of hepatitis C virus infection. Eur J Gastroenterol Hepatol 2006 September; 18(9):991-997), cytokines (WO 00/08215), chemokines and interleukins as well as interferon-stimulated genes (Yee L J, Perez K A, Tang J, van Leeuwen D J, Kaslow R A. Association of CTLA4 polymorphisms with sustained response to interferon and ribavirin therapy for chronic hepatitis C virus infection. J Infect Dis 2003 Apr. 15; 187(8):1264-1271; Goulding C, McManus R, Murphy A, MacDonald G, Barrett S, Crowe J, et al. The CCR5-delta32 mutation: impact on disease outcome in individuals with hepatitis C infection from a single source. Gut 2005 August; 54(8): 1157-1161; Uzé G, Monneron D. IL-28 and IL-29: newcomers to the interferon family. Biochimie 2007 June-July; 89(6-7):729-34; Kwoh et al., 1990, Am. Biotechnol. Lab. 8:14-25; Hagen-Mann, et al., 1995 Exp. Clin. Endocrinol. Diabetes 103: 150-155) on HCV infection outcomes.

Previous studies have used a candidate gene approach based on a priori knowledge of the potential role of a gene in HCV infection. However, previous data do not allow accurate prediction of spontaneous clearance or response to treatment (Kau A, Vermehren J, Sarrazin C. Treatment predictors of a sustained virologic response in hepatitis B and C. J Hepatol 2008 October; 49(4):634-651). In 2009, several groups reported results from independent genome-wide association studies (GWAS) that identified single nucleotide polymorphisms (SNPs) in the IL28 gene region that are associated with response to pegylated IFN-α/ribavirin treatment among patients with chronic hepatitis C, as well as spontaneous clearance of HCV infection. For example, U.S. Patent Publication No. US2011/0165124 by Bochud et al, which is incorporated herein in its entirety by reference, discloses numerous SNPs associated with both response to interferon-based treatment of HCV, and spontaneous clearance. Among the SNPs identified in these GWAS, genotype based on rs12979860 is currently accepted as the best predictor of spontaneous clearance and treatment response. In fact, there are currently available two laboratory tests based on detection of this polymorphism. While these tests have proved to be useful in identifying responders to treatment of chronic HCV infection, there remains a need for a more robust and accurate test for predicting spontaneous clearance and response to treatment. Compared to persons of European ancestry, African American patients have a higher frequency of chronic hepatitis C and a poorer response to therapy with IFN-α/ribavirin. Racial differences in the frequency of GWAS marker rs12979860 do not completely explain these disparities. Identification of a genetic marker that has optimal predictive values in all population groups would improve clinical decision models for treatment of chronic hepatitis C and help deliver ‘personalized medicine’ to all HCV-infected patients.

The present invention satisfies this need and provides other benefits as well.

### SUMMARY OF THE INVENTION

One aspect of the invention is a method for predicting the ability of an individual to spontaneously clear an HCV infection by obtaining a biological sample from the individual, analyzing the sample to identify which alleles of the ss469415590 polymorphism are present in the individual and selecting individuals predicted to spontaneously clear the HCV infection if the ss469415590 insertion allele is present in the individual, or selecting the individual as unable to spontaneously clear the HCV infection if the ss469415590 insertion allele is absent in the individual.

A related aspect of the invention is a method for predicting the ability of an individual to spontaneously clear an HCV infection by obtaining a biological sample from the individual, analyzing the sample to identify which alleles the ss469415590 polymorphism are present in the individual, selecting individuals as predicted to be unable to spontaneously clear the HCV infection if the ss469415590 deletion allele is present in the individual; or, selecting the individual as being predicted to spontaneously clear the HCV infection if the ss469415590 deletion allele is absent in the individual.

Another aspect of the invention is a method for predicting the spontaneous clearance of HCV infection in an individual suffering from a hepatitis C infection by obtaining a biological sample from the individual, detecting in the biological sample the presence of at least one polymorphism from the group including:
i) deletion of the nucleotide corresponding to position 27 of SEQ ID NO:1;
ii) a substitution of the thymidine corresponding to position 28 of SEQ ID NO:1 with guanine, adenine or cytosine; and,
iii) a deletion of the nucleotide corresponding to position 27 of SEQ ID NO:1, and a substitution of the thymidine corresponding to position 28 of SEQ ID NO:1 with guanine, adenine or cytosine.
   If at least one polymorphism is detected, the individual is selected as having a decreased likelihood of spontaneously clearing the HCV present.

In related embodiments, these methods are conducted on individuals infected with HCV.

In other embodiments, these methods on conducted on individuals who are not infected with HCV.

In related embodiments, the likelihood of the individual spontaneously clearing the HCV infection has an odds ratio of at least 1.2.

Another embodiment is a method for predicting the clinical response of an individual suffering from an HCV infection to administration of a therapeutic treatment by obtaining a biological sample from the individual, analyzing the sample to identify which alleles the ss469415590 polymorphism are present in the individual, selecting the individual as predicted to respond to the administration of the therapeutic treatment, if the ss469415590 insertion allele is present in the individual; or, selecting the individual as predicted to not respond to the administration of the therapeutic treatment, if the ss469415590 insertion allele is absent in the individual.

Another embodiment is a method for predicting the clinical response of an individual suffering from an HCV infection to administration of a therapeutic treatment by obtaining a biological sample from the individual, analyzing the sample to identify which alleles the ss469415590 polymorphism are present in the individual, selecting the individual as predicted to not respond to the administration of the therapeutic treatment, if the ss469415590 deletion allele is present in the individual; or, selecting the individual as predicted to respond to the administration of the therapeutic treatment, if the ss469415590 deletion allele is absent in the individual.

Another embodiment is a method for predicting the clinical response of an individual suffering from HCV infection to administration of a therapeutic treatment, the method comprising obtaining a biological sample from the individual, detecting in the biological sample the presence of at least one polymorphism from the group of:
i) deletion of the nucleotide corresponding to position 27 of SEQ ID NO:1;
ii) a substitution of the thymidine corresponding to position 28 of SEQ ID NO:1 with guanine, adenine or cytosine; and,
iii) a deletion of the nucleotide corresponding to position 27 of SEQ ID NO:1, and a substitution of the thymidine corresponding to position 28 of SEQ ID NO:1 with guanine, adenine or cytosine;
   If at least one polymorphism is present, the individual is predicted to be unable to respond to the administration of the therapeutic treatment, or, if the at least one polymorphism is absent, the individual is predicted to respond to the administration of the therapeutic treatment.

In some embodiments of these methods the likelihood of the individual responding to treatment has an odds ratio of at least 1.2.

In specific embodiments of these methods, the treatment comprises interferon. In some embodiments, the interferon is selected from the group consisting of IFN-α, IFN-λ, and pegylated-IFN.

In specific embodiments of these methods, the treatment comprises ribavirin.

In specific embodiments of these methods, the treatment comprises a direct acting antiviral agent.

In some embodiments of these methods, the HCV infection is an acute infection.

In some embodiments of these methods, the HCV infection is a chronic infection.

In some embodiments of these methods, the step of selecting is performed using a device, such as an electronic device.

One embodiment is a method of treating a patient suffering from a chronic HCV infection by obtaining a biological sample comprising nucleic acid molecules from the patient, detecting in the biological sample the presence of at least one polymorphism selected from the group of:
i) deletion of the nucleotide corresponding to position 27 of SEQ ID NO:1;
ii) a substitution of the thymidine corresponding to position 28 of SEQ ID NO:1 with guanine, adenine or cytosine; and,
iii) a deletion of the nucleotide corresponding to position 27 of SEQ ID NO:1, and a substitution of the thymidine corresponding to position 28 of SEQ ID NO:1 with guanine, adenine or cytosine.
   A therapeutic dose of ribavirin and an interferon is administered to the patient if the at least one polymorphism is absent in the sample, or, the dose of ribavirin and interferon is withheld if the at least one polymorphism is present in the sample.

In these methods, the interferon administered may be selected from the group of IFN-α, IFN-λ, and pegylated-IFN.

In these methods, the interferon may be combined with a drug selected from the group consisting of ribavirin, an anti-protease drug, an additional antiviral drug, and combinations thereof.

In these methods, the biological sample is selected from the group consisting of blood, saliva, urine, a skin scraping, a tissue sample and a buccal swab.

In these methods, the step of detecting may comprise at least one technique selected from the group of:
a) sequence determination of at least a portion of a nucleic acid molecule corresponding to chromosome 19 represented by nucleotides 26-29 of SEQ ID NO:1;
b) polymerase chain reaction; and
c) nucleic acid hybridization.

Another embodiment is a kit useful for predicting the ability of an individual to spontaneously clear hepatitis C virus. The kit comprises at least one reagent for performing the methods of detecting the presence of at least one polymorphism from the group including:
i) deletion of the nucleotide corresponding to position 27 of SEQ ID NO:1;
ii) a substitution of the thymidine corresponding to position 28 of SEQ ID NO:1 with guanine, adenine or cytosine; and,
iii) a deletion of the nucleotide corresponding to position 27 of SEQ ID NO:1, and a substitution of the thymidine corresponding to position 28 of SEQ ID NO:1 with guanine, adenine or cytosine, and instructions for using the at least one reagent.

Similarly, a related kit of the invention is useful for predicting the clinical response of an individual suffering from an HCV infection to administration of a therapeutic treatment. The kit comprises at least one reagent for performing the methods of detecting the presence of at least one polymorphism from the group including:
i) deletion of the nucleotide corresponding to position 27 of SEQ ID NO:1;
ii) a substitution of the thymidine corresponding to position 28 of SEQ ID NO:1 with guanine, adenine or cytosine; and,
iii) a deletion of the nucleotide corresponding to position 27 of SEQ ID NO:1, and a substitution of the thymidine corresponding to position 28 of SEQ ID NO:1 with guanine, adenine or cytosine, and instructions for using the at least one reagent.

### BRIEF DESCRIPTION OF THE FIGURES

FIG. 1. Identification of a novel transcribed region upstream of IL28. a. RNA-seq in primary human hepatocytes (PHH) treated with 50 ug/ml of PolyI:C for 0, 1, 2, 4, 8 or 24 hours. RNA-seq plot of the 150-Kb region shows expression of the IFN-λ genes (IL28B, IL28A and IL29) and a novel transcribed region upstream of IL28B. The results are presented as clusters of RNA-seq reads, with the number of reads (depth) corresponding to the level of mRNA expression. b. Detailed view of the RNA-seq results for IL28B gene and the novel transcribed region. A CTCF transcriptional insulator (based on ENCODE data from the UCSC Genome Browser) separates these transcription units, indicating their independence.

FIG. 2. DNA sequence analysis of the regions upstream of the IL28A and IL28B genes. Identical nucleotides are shaded in black. The location of ss469415590 is marked by a heavy, black bar.

FIG. 3. Genotyping of ss469415590 polymorphism using a custom-designed TaqMan genotyping assay

FIG. 4. Results of Sanger sequencing using DNA from individuals having different alleles of the ss469415590 polymorphism.

FIG. 5. Allele frequencies for ss469415590 and GWAS markers rs12979860 and rs8099917 in HapMap populations reference panels. rs8099917 genotypes were downloaded from HapMap website, while ss469415590 and rs12979860 were genotyped in all Hapmap samples. Pair-wise linkage disequilibrium estimates (r²) are between ss469415590 and other markers.

FIG. 6. SNPs from a 100-Kb genomic region in high linkage disequilibrium with ss469415590 (r²>0.6 in YRI or r²>0.75 in other populations) in the 1000 Genomes Project reference panel (obtained from 1000 genomes website), October 2010 release. In the 1000 Genomes reference panel ss469415590 (TT/ΔG) is represented by rs74597329 (T/G). The GWAS marker rs12979860 is shown in bold.

FIG. 7. Characteristics of the Virahep-C and HALT-C participants

FIG. 8. Characteristics of the UHS and ALIVE participants.

FIG. 9. Median decrease in HCV RNA (log₁₀ IU/ml) in African-American participants in Virahep-C study during the first 28 days of treatment with pegIFN-α/RBV. p=0.015 for comparison of mean differences in HCV RNA levels at day 28 for each of the three genotype groups for ss469415590 with the respective group for rs12979860.

FIG. 10. Comparison of rs12979860 and ss469415590 genotypes for predicting the median decrease in HCV RNA load (log₁₀ IU/ml) after 28 days of treatment with pegIFN-a/RBV in European-American (EA) and African-American (AA) participants in Virahep-C. Analysis is limited to subjects successfully genotyped for both ss469415590 and rs12979860.

FIG. 11. Comparison of rs12979860 and ss469415590 genotypes for predicting 24-week, end-of-treatment and sustained virological response (SVR) to pegIFN-α/RBV treatment in European-American (EA) and African-American (AA) participants in Virahep-C. Analysis is limited to subjects successfully genotyped for both ss469415590 and rs12979860.

FIG. 12. Comparison of rs12979860 and ss469415590 genotypes for predicting 20-weeks, end-of-treatment and sustained virological response (SVR) to pegIFN-α/RBV treatment in European-American (EA) and African-American (AA) participants enrolled in the HALT-C Trial. Analysis is limited to subjects successfully genotyped for both ss469415590 and rs12979860.

FIG. 13. Association of the GWAS marker rs12979860 and ss469415590 with spontaneous clearance of hepatitis C virus infection among European-American and African-American injection drug users enrolled in the Urban Health Study (UHS). Analysis is limited to subjects successfully genotyped for both ss469415590 and rs12979860.

FIG. 14. Association of the GWAS marker rs12979860 and ss469415590 with spontaneous clearance of hepatitis C virus infection among African-American injection drug users enrolled in ALIVE study. Analysis is limited to subjects successfully genotyped for both ss469415590 and rs12979860.

FIG. 15. Sequence and genetic variants in the area surrounding ss469415590.

FIG. 16. Analysis of allele frequencies of all variants surrounding ss469415590 based on Sanger sequencing and TaqMan genotyping of HapMap samples. (270 individuals in total). Markers observed polymorphic more than in one individual at least in one population are presented.

FIG. 17. Analysis of pair-wise linkage disequilibrium (r2) within IL28B upstream region in HapMap samples from Europeans (CEU) (a), West-Africans (YRI) (b) and Asians-Chinese (CHB) and Japanese (JPT) (c). Monomorphic markers are highlighted in light-gray.

FIG. 18. Haplotype analysis in the IL28B region in HapMap samples and participants of Virahep C study. Sustained virological response (SVR) was used as pegIFN-α/RBV therapy treatment outcome (responders/non-responders). Bold underlined are markers included in the haplotype analysis of SVR by Smith et. al, 2011., *—8 markers used in the final haplotype analysis in HapMap and Virahep-C samples. In yellow—the unfavorable haplotypes based on studies in Europeans and Asians, and their extrapolation in Africans; in grey—a common favorable haplotype shared by all populations. EA—European-Americans; AA—African-Americans. Haplotype frequencies in all groups (HapMap and responders/non-responders) are indicated as %. Highlighted bold underlined are variants with possible modifying effects on risk haplotypes—ss539198934, rs137902769, rs117648444, rs142981501 and rs73555604.

### DETAILED DESCRIPTION OF THE INVENTION

The present invention generally relates to methods of identifying individuals who are most likely to spontaneously clear an HCV infection. It also relates to methods of identifying HCV-infected individuals who are most likely to respond to, and thus benefit from, therapeutic treatment for an HCV infection. More specifically, the present invention relates to the discovery that the detection of specific genetic polymorphisms can be used to determine the likelihood of an individual responding to an HCV treatment, or the likelihood of an HCV infected individual to spontaneously clear an HCV infection. In this regard, the presence of particular alleles of such polymorphisms in an individual relates to the ability of the individual to spontaneously clear an HCV infection, and the likelihood of the individual to respond to treatment for an HCV infection.

The inventors have discovered a novel polymorphism referred to as ss469415590 {NCBI reference number NC_{—}000019.9:[g.39739154delT;g.39739155T>G]). The ss469415590 polymorphism consists of two nucleotide variations, one of which is present in variants of the rs67272382 polymorphism, the other of which is present in variants of the rs74597329 polymorphism. The rs74597329 polymorphism is in complete linkage with rs67272382 in all populations studied (i.e., Europeans, Africans, Asians) and thus, for simplicity only the compound ss469415590 polymorphism will be referred to. However, it should be understood that for purposes of the present invention, detection of alleles of any polymorphism selected from the group consisting of rs67272382, and rs74597329 can provide the same genetic information as ss469415590.

The inventors have found that the presence of a particular allele of the ss469415590 polymorphism in a nucleic acid sample from an individual suffering from chronic hepatitis indicates the individual has an increased likelihood of responding to a therapeutic treatment for HCV infection. The inventors have also found that the presence of a particular allele of the ss469415590 polymorphism in a nucleic acid sample from an individual indicates that the individual has an increased likelihood of spontaneously clearing an HCV infection. The present methods are also useful for prescribing a treatment, such as an HCV treatment, to an individual in need thereof, who would benefit from such treatment.

The methods of the present invention may generally be accomplished by obtaining a biological sample from an individual, and analyzing the sample in order to identify the allele(s) of the ss469415590 polymorphism carried by the individual.

The presence of a particular allele of the ss469415590 polymorphism indicates that the individual is more likely to spontaneously clear an HCV infection, and is more likely to respond to a therapeutic treatment for HCV, than is an individual lacking the particular allele. Moreover, the presence of a particular, alternative allele of the ss469415590 polymorphism indicates that the individual is less likely to spontaneously clear an HCV infection, and is less likely to respond to a therapeutic treatment for HCV, than is an individual lacking the particular, alternative allele.

Accordingly, one embodiment of the present invention is a method for predicting the likelihood of an individual to spontaneously clear an HCV infection, the method comprising:
a) obtaining a biological sample from the individual;
b) analyzing the sample to identify which allele(s) the ss469415590 polymorphism is/are present at a locus in chromosome 19 represented by SEQ ID NO:5 or SEQ ID NO:7;
wherein the presence of a particular allele of the ss469415590 polymorphism indicates the likelihood of the individual spontaneously clearing an HCV infection.

In one embodiment, the presence of an rs67272382 insertion allele indicates the individual is predicted to spontaneously clear a HCV infection. In one embodiment, the presence of an ss469415590 insertion allele indicates that the individual is predicted to spontaneously clear an HCV infection. In one embodiment, the presence of an rs67272382 deletion allele indicates that the individual is predicted to be unable to spontaneously clear an HCV infection. In one embodiment, the presence of an ss469415590 deletion allele indicates the individual is predicted to be unable to spontaneously clear an HCV infection.

Another embodiment of the present invention is a method for predicting the likelihood that an individual will respond to a treatment for HCV infection, the method comprising:
a) obtaining a biological sample from the individual;
b) analyzing the sample to identify which allele(s) of at least one polymorphism selected from the group consisting of the rs67272382 polymorphism and the ss469415590 polymorphism, is present at a locus in chromosome 19 represented by SEQ ID NO:1, SEQ ID NO:3, SEQ ID NO:5 or SEQ ID NO:7;
wherein the presence of a particular allele of the at least one polymorphism indicates the likelihood the individual will respond to treatment for an HCV infection.

In one embodiment, the presence of an rs67272382 insertion allele indicates the individual is predicted to respond to treatment for an HCV infection. In one embodiment, the presence of an ss469415590 insertion allele indicates that the individual is predicted to respond to treatment for an HCV infection. In one embodiment, the presence of an rs67272382 deletion allele indicates that the individual is predicted to not respond to treatment for an HCV infection. In one embodiment, the presence of an ss469415590 deletion allele indicates the individual is predicted to not respond to treatment for an HCV infection.

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

Unless defined otherwise, all technical and scientific terms used herein have the same meaning as is commonly understood by one of skill in art to which the subject matter herein belongs. As used herein, the following definitions are supplied in order to facilitate the understanding of the present invention.

It is to be noted that the term “a” “an” “one or more” and “at least one” can be used interchangeably herein. The terms “comprising,” “including,” and “having” can also be used interchangeably. Furthermore, the phrase “selected from the group consisting of” refers to one or more members of the group in the list that follows, including mixtures (i.e. combinations) of two or more members. As used herein, “at least one” means one or more. The term “comprise” is generally used in the sense of “including”, that is to say “permitting the presence of one or more features or components”. It is to be further understood that where descriptions of various embodiments use the term comprising, those skilled in the art would understand that in some specific instances, an embodiment can be alternatively described using language “consisting essentially of”.

The terms individual, subject, and patient are well-recognized in the art, and are herein used interchangeably to refer to a mammal, including dog, cat, rat, mouse, monkey, cow, horse, goat, sheep, pig, camel, and, most preferably, a human. In some embodiments, the subject is in need of a hepatitis C treatment. For example, in one embodiment the subject is infected with HCV. However, in other embodiments, the subject is not infected with HCV. In one embodiment, the subject is at risk for infection with HCV. In one embodiment, the subject has been exposed to HCV. As used herein, the terms exposed, exposure, and the like, indicate the subject has come in contact with bodily fluid from another individual who is infected with HCV. Contact can occur through such things as, for example, a needle stick, sexual contact, or the birthing process.

The terms individual, subject, and patient by themselves do not denote a particular age, sex, race, and the like. Thus, individuals of any age, whether male or female, are intended to be covered by the present disclosure. Likewise, the methods of the present invention can be applied to any race, including, for example, Caucasian (white), African-American (black), Native American, Native Hawaiian, Hispanic, Latino, Asian, and European. In some embodiments of the present invention, such characteristics are significant. In such cases, the significant characteristic(s) (age, sex, race, etc.) will be indicated.

The term hepatitis C virus, or HCV, is used herein to define an RNA viral species of which pathogenic strains cause hepatitis C, also known as non-A, non-B hepatitis. Based on genetic differences between HCV isolates, the hepatitis C virus species is classified into six major genotypes (1-6) with several subtypes within each genotype. Subtypes are further broken down into quasi species based on their genetic diversity. The preponderance and distribution of HCV genotypes varies globally. For example, in North America, genotype 1a predominates followed by 1b, 2a, 2b, and 3a. In Europe, genotype 1b is predominant followed by 2a, 2b, 2c, and 3a. Genotypes 4 and 5 are found almost exclusively in Africa. The viral genotype may be clinically important in determining potential response to interferon-based therapy and the required duration of such therapy. Genotypes 1 and 4 are generally less responsive to interferon-based treatment than are the other genotypes (2, 3, 5 and 6). It is to be noted that genotypes 5 and 6 are rare in the U.S. population.

As used herein, hepatitis C is an infectious disease affecting the liver, which is caused by the hepatitis C virus (HCV). The initial infection with HCV may produce acute symptoms or the individual may be asymptomatic (without symptoms), but once established, chronic hepatitis C infection can progress to scarring of the liver (fibrosis), and advanced scarring (cirrhosis) which is generally apparent after many years. In some cases, those with cirrhosis will go on to develop liver failure or other complications of cirrhosis, including liver cancer.

According to the present invention, chronic hepatitis C refers to an infection with HCV that persists for more than six months. Clinically, it is often asymptomatic and it is often discovered accidentally. The natural course of chronic hepatitis C varies considerably from one person to another. Although almost all people infected with HCV have evidence of inflammation on liver biopsy, the rate of progression of liver scarring (fibrosis) shows significant variability among individuals. Accurate estimates of the risk over time are difficult to establish because of the limited time that tests for this virus have been available.

In some embodiments, the individual is co-infected with at least one other organism such as, for example, hepatitis B virus, hepatitis A virus, staphylococcus aureus, and/or the human immunodeficiency virus (HIV).

As used herein, spontaneous clearance refers to the ability of an infected individual to clear HCV from their blood without the need for administration of a therapeutic treatment designed to aid such clearance. If an individual is capable of spontaneously clearing an HCV infection, such clearance is typically observed during an acute infection. Authoritative clinical reviews have generally quoted clearance rates as low as 10-15%. Methods of measuring the levels of HCV are known to those skilled in the art. For example, one method of determining the level of HCV in an infected individual is to measure the amount of HCV RNA present in the individual's blood. Such measurement can be made using any known method for detecting RNA, such as, for example, the use of nucleic acid binding dyes, PCR amplification and/or nucleic acid hybridization.

As used herein, the terms treat, treatment, and the like, refer to therapeutic treatment and prophylactic treatment, or preventative measures, wherein the object is to prevent or slow down (lessen) an undesired physiological condition or disease, or obtain beneficial or desired clinical results. In this regard, treatment refers to the administration of a therapeutic agent to slow down or prevent an undesired physiological condition or disease, or the symptoms associated with the above conditions or diseases. In one embodiment, the treatment is one that helps a patient responsive to such treatment reduce the level of HCV RNA present in their body. By association, such a reduction reflects a reduction in the level of HCV present in the patient's body. In one embodiment, the treatment reduces the amount of HCV RNA in the patient's body by at least 50%, at least 75%, at least 85%, at least 95%, or at least 99% during the course of treatment. In one embodiment, treatment completely eliminates HCV RNA from the patient.

With regard to treatments for HCV, methods of the present invention can be used to predict the response to any therapeutic agent useful for treating HCV infections. Generally, treatment for an HCV infection is an interferon based treatment. Thus, in one embodiment, treatment is an interferon-based treatment. In various preferred embodiments, the interferon-based treatment is selected from the group comprising IFN-α, IFN-λ or any pegylated-interferon. In another embodiment, the interferon-based treatment is combined with ribavirin. In various embodiments, further combinations can include antiprotease drugs and/or other antiviral drugs. Thus, one embodiment of the present invention is a method for predicting the likelihood that an individual will respond to a treatment for HCV infection, the method comprising:
a) obtaining a biological sample from the individual;
b) analyzing the sample to identify which allele(s) of at least one polymorphism selected from the group consisting of the rs67272382 polymorphism and the ss469415590 polymorphism, is present at a locus in chromosome 19 represented by SEQ ID NO:1, SEQ ID NO:3, SEQ ID NO:5 or SEQ ID NO:7;
wherein the presence of a particular allele of the at least one polymorphism indicates the likelihood the individual will respond to treatment for an HCV infection using IFN-α, IFN-λ, pegylated-interferon, anti-protease drugs, anti-viral drugs, or combinations thereof.

In one embodiment, the presence of an rs67272382 insertion allele indicates the individual is predicted to respond to treatment for an HCV infection using IFN-α, IFN-λ, pegylated-interferon, anti-protease drugs, anti-viral drugs, or combinations thereof. In one embodiment, the presence of an ss469415590 insertion allele indicates that the individual is predicted to respond to treatment for an HCV infection using IFN-α, IFN-λ, pegylated-interferon, anti-protease drugs, anti-viral drugs, or combinations thereof. In one embodiment, the presence of an rs67272382 deletion allele indicates that the individual is predicted to not respond to treatment for an HCV infection using IFN-α, IFN-λ, pegylated-interferon, anti-protease drugs, anti-viral drugs, or combinations thereof. In one embodiment, the presence of an ss469415590 deletion allele indicates the individual is predicted to not respond to treatment for an HCV infection using IFN-α, IFN-λ, pegylated-interferon, anti-protease drugs, anti-viral drugs, or combinations thereof.

While a treatment for an HCV infection can be administered to help a patient clear an HCV infection, not all patients are responsive to such treatment. That is, in some patients, while the treatment may cause some reduction in the level of HCV RNA, it does not result in a sustained virological response. A patient for whom treatment does not result in a sustained virological response is referred to as a non-responder. Likewise, a patient for whom treatment results in a sustained virological response is referred to as a responder. A sustained virological response is defined as the lack of HCV RNA in the blood 24 weeks after the cessation of treatment. In determining such a result, the HCV RNA level is typically measured at several times points during the course of treatment in order to measure the treatment response. The lower the HCV RNA level is at these time points, the more likely it is that a patient will achieve a sustained virological response.

As used herein, predicting a clinical response refers to knowing the likelihood that a patient will spontaneously clear an HCV infection prior to, or during, the acute phase of infection with HCV. It also refers to knowing the likelihood that a treatment for HCV infection will cause a sufficient reduction in the level of HCV RNA in a patient, before such treatment is administered to the patient. With regard to the present invention, predicting the clinical response may also be referred to as determining the susceptibility of a patient to response, or non-response, to a treatment for HCV infection, or the susceptibility of a patient to spontaneously clearing the virus.

As used herein the terms susceptible, susceptibility, and the like, refer to the likelihood, or probability, an individual will spontaneously clear an HCV infection, or will respond to treatment for such an infection. Such likelihood can also be referred to as a predisposition. In the context of the present invention, the likelihood of spontaneously clearing an HCV infection and/or responding to a treatment need not be absolute. That is, for example, while the presence of a particular allele of the rs67272382 polymorphism, or the ss469415590 polymorphism, increases the likelihood that a patient will spontaneously clear an HCV infection, or respond to treatment, in a population of patients, all of whom carry such allele(s), some percentage of such population may not spontaneously clear an HCV infection or respond to treatment. This is likely due to a combination of other factors such as, for example, the genotype of the virus, race, age, gender, and the genetic makeup of the individual at loci other than those in the IL28B region of the genome. Thus, in one embodiment, the presence of a particular allele of at least one polymorphism selected from the group consisting of rs67272382 and ss469415590, indicates an individual is more likely to spontaneously clear an HCV infection than is a patient not having the particular allele(s). In one embodiment, the presence of a particular allele of at least one polymorphism selected from the group consisting of rs67272382 and ss469415590, indicates an individual is more likely to respond to treatment for an HCV infection than is a patient not having the particular allele(s). Thus, a patient having such particular allele(s) is more likely to benefit from administration of a treatment than is a patient not having the particular allele(s). In other embodiments, the presence of an particular, alternative allele of a polymorphism selected from the group consisting of rs67272382 and ss469415590, indicates an individual is less likely to spontaneously clear an HCV infection than is a patient not having the particular, alternative allele(s). In yet another embodiment, the presence of an particular, alternative allele of a polymorphism selected from the group consisting of rs67272382 and ss469415590, indicates an individual is less likely to respond to a treatment for an HCV infection than is a patient not having the particular, alternative allele(s). Thus, a patient having such particular, alternative allele(s) is less likely to benefit from administration of a treatment than is a patient not having the particular.

Thus, it will be appreciated by those skilled in the art that, likelihood, susceptibility, predisposition, and the like, are relative terms. Methods of quantifying and reporting the likelihood of a patient to respond to treatment or spontaneously clear an HCV infection are known to those skilled in the art. For example, one such method is a relative indication determined by comparing the number of patients having a particular allele of the rs67272382 polymorphism and/or the ss469415590 polymorphism, and that spontaneously clear an HCV infection, with the number of patients lacking such allele(s) and that also spontaneously clear HCV infection. A similar comparison can be made between people that do, or do not have specific alleles of the rs67272382 polymorphism and/or the ss469415590 polymorphism, and who respond to treatment for an HCV infection. Such a relative comparison can be illustrated using a fold increase; for example, 1.5 fold (1.5×), 2×, 3×, 5×, etc. Such relative comparison can also be illustrated using a percent increase. For example, if the number of patients having at least one polymorphism of the present invention and that respond to treatment or spontaneously clear HCV is twice the number of patients that lack such polymorphisms and that respond to treatment or spontaneously clear the virus, it could be said that patients having at least one such polymorphism are 100% more likely to respond to treatment or spontaneously clear HCV

Relative comparisons can also be illustrated using an odds ratio, which is a statistical method for relative comparisons that is used when selection of study subjects is based on the clinical outcome of interest. In one embodiment, the likelihood of an individual spontaneously clearing an HCV infection, or responding to a treatment for HCV, has an odds ratio of at least about 1.2, at least about 1.4, at least about 1.6, at least about 1.8, at least about 2.0, at least about 2.2, at least about 2.4, at least about 2.6, at least about 2.8, at least about 3.0, at least about 3.2, at least about 3.4, at least about 2.6, at least about 3.8, at least about 4.0, at least about 4.2, at least about 4.4, at least about 4.6, at least about 4.8 or at least about 5.0. Methods of calculating an odds ratio are known to those skilled in the art and are exemplified in Rothman, Kenneth J.; Greenland, Sander; Lash, Timothy L. (2008). Modern Epidemiology. Lippincott Williams & Wilkins. Third edition, which is incorporated herein in its entirety.

As used herein, a biological sample refers to any fluid or tissue from an individual that can be analyzed for the presence of a polymorphism. Preferably, such a sample comprises nucleic acid molecules. In one embodiment, the sample comprises DNA. In one embodiment the sample comprises cDNA. In one embodiment, the sample comprises RNA. RNA can be one or more of mRNA and mi RNA. In one embodiment, the RNA is mRNA. In one embodiment, the RNA is miRNA. Examples of the type of sample that can be used to practice the present invention include, but are not limited to, a blood sample, a urine samples, a tear sample, a tissue sample, and a buccal swab. Samples useful for detecting the presence of a polymorphism are known to those skilled in the art. Moreover, methods of obtaining such samples are also known to those skilled in the art.

Once a sample has been obtained, it is analyzed for the presence or absence of specific alleles of polymorphisms of the present invention. In the case of a method of determining the likelihood of a response to a treatment for an HCV infection, or resistance to such treatment, according to the present invention, the presence of a specific allele of a polymorphism of the present invention indicates the likelihood of the individual to respond to such treatment. In the case of a method of determining the likelihood of spontaneously clearing an HCV infection, according to the present invention, the presence of a specific allele of a polymorphism of the present invention indicates the likelihood of the individual spontaneously clearing an HCV infection.

As used herein, polymorphism refers to the occurrence in a population of two or more alternative sequences at a specific location in a chromosome. That is, a polymorphism refers to a site in a chromosome having an alternative nucleotide sequence when compared to the same site in the homologous chromosome from the same individual or from a different individual. Such sequence differences result from deletion, insertion or substitution of nucleotides. A polymorphism may comprise one or more base changes, insertions, repeats, or deletions. A polymorphism may be as small as one base pair. Such a polymorphism is referred to as a single nucleotide polymorphism (SNP). A polymorphism may also consist or comprise of more than one nucleotide. Such a polymorphism is referred to as a compound polymorphism. A polymorphism consisting of two nucleotide positions is referred to as a dinucleotide polymorphism. Polymorphisms of the present invention include variable number of tandem repeats (VNTR's), hypervariable regions, minisatellites, dinucleotide repeats, trinucleotide repeats, tetra- and more nucleotide repeats, simple sequence repeats, copy number variations (CNV), insertion elements such as Alu, insertions, deletions and substitutions. A polymorphism may fall within a coding sequence or it may fall within a non-coding sequence. Moreover, a polymorphism that falls within a coding sequence may or may not affect the sequence of the encoded protein. Preferred polymorphisms of the present invention have at least two alleles, each occurring at a frequency of preferably greater than 1%, more preferably greater than 5%, more preferably greater than 10%.

As used herein, an allele refers to one specific form of a polymorphism. If a specific sequence contains a polymorphism having several sequence variations, each unique variation is referred to as an allele. For example, if a particular position in a nucleotide sequence in a chromosome contains a cytosine, and the corresponding position in the homologous chromosome contains a thymidine, such a polymorphism is said to have two alleles. If a third form of the chromosome exists in which the corresponding position is a guanine, the polymorphism would be said to have three alleles. Moreover, as an example of how such alleles can be identified, or differentiated from one another, the exemplified alleles can be referred to as a C allele, a T allele and G allele, respectively. The specific nucleotide changes at these variant sites that differ between different alleles are termed variants, polymorphisms, or mutations.

One allelic form of a polymorphism may be arbitrarily designated as the reference form and other allelic forms are designated as alternative or variant alleles. For example, if a particular allele is associated with a particular phenotypic characteristic (e.g., the presence of a disease, the ability to respond to a disease, the ability to respond to treatment for a disease, etc.), the beneficial allelic form may be referred to as a “wild-type form” or beneficial form, while the unfavorable, disease-associated allelic form can be referred to as the disadvantageous form, the unfavorable form, the mutant form, the alternative form, the genetic risk variant, and the like.

A diallelic polymorphism has two forms. A triallelic polymorphism has three forms. A polymorphism that is inherited from generation to generation and can be found in every cell of the body is called a germline genetic variant. In one embodiment, polymorphisms of the present invention are germline genetic variants. Other variants are called somatic mutations because they can be found only in some cells of the body and are not heritable. Somatic variants can be caused by exposure to or contact with chemicals, enzymes, or other agents, or exposure to agents that cause damage to nucleic acids, for example, ultraviolet radiation, mutagens or carcinogens. A particular kind of polymorphism, called a single nucleotide polymorphism, or SNP, is a small genetic change or variation that can occur within a person's DNA sequence.

As has been described, the present invention relates to the detection of alleles of a polymorphism selected from the group consisting of rs67272382 and ss469415590. According to the present invention, each of these polymorphisms has at least two alleles. With regard to the rs67272382 polymorphism, one allele, referred to as the rs67272382 insertion allele, is represented by SEQ ID NO:1 A variant of this allele (i.e., an alternative allele), which is referred to as the rs67272382 deletion allele, is represented by SEQ ID NO:3. It can be seen from examination of these sequences that these two alleles differ in that the thymidine at position 27 in the rs67272382 insertion allele has been deleted in the rs67272382 deletion allele. With regard to the ss469415590 polymorphism, one allele, referred to as the ss469415590 insertion allele, is represented by SEQ ID NO:5. A variant of this allele (i.e., an alternative allele), which is referred to as the ss469415590 deletion allele (also referred to as AG), is represented by SEQ ID NO:7. It can be seen from examination of these sequences that these two alleles differ in that one of the thymidines corresponding to the thymidine at position 27 in the ss469415590 insertion allele has been deleted in the ss469415590 deletion allele. In addition, the remaining thymidine (i.e., position 27) has been substituted with a guanine.

Thus, one embodiment of the present invention is a method for predicting the likelihood of an individual to spontaneously clear an HCV infection, the method comprising:
a) obtaining a biological sample from the individual;
b) analyzing the sample to determine if at least one allele selected from the group consisting of the rs67272382 insertion allele and the ss469415590 insertion allele, is present at a locus in chromosome 19 represented by SEQ ID NO:1 or SEQ ID NO:5;
wherein the presence of the rs67272382 insertion allele and/or the ss469415590 insertion allele indicates the individual is more likely to clear an HCV infection than is an individual lacking either insertion allele.
   In one embodiment, an individual having the rs67272382 insertion allele and/or the ss469415590 insertion allele is at least 1.5× (fold), 2.0×, 2.5×, 3.0×, 4.0×, or 5.0× more likely to clear an HCV infection than is an individual lacking either insertion allele. Thus, for example, if an individual is determined to be 2× more likely to spontaneously clear an HCV infection, such a relative number means that in a population representative of the individual, it would be expected that twice as many individuals would spontaneously clear an HCV infection as the number if individual that would not spontaneously clear an HCV infection. In one embodiment, the likelihood that an individual having the rs67272382 insertion allele and/or the ss469415590 insertion allele will respond to treatment for an HCV infection has an odds ratio of at least about 1.2, at least about 1.4, at least about 1.6, at least about 1.8, at least about 2.0, at least about 2.2, at least about 2.4, at least about 2.6, at least about 2.8, at least about 3.0, at least about 3.2, at least about 3.4, at least about 2.6, at least about 3.8, at least about 4.0, at least about 4.2, at least about 4.4, at least about 4.6, at least about 4.8 or at least about 5.0. In one embodiment, the individual is predicted to be able to spontaneously clear an HCV infection.

One embodiment of the present invention is a method for predicting the likelihood that an individual will respond to a treatment for HCV infection, the method comprising:
a) obtaining a biological sample from the individual;
b) analyzing the sample to determine if at least one allele selected from the group consisting of the rs67272382 insertion allele and the ss469415590 insertion allele, is present at a locus in chromosome 19 represented by SEQ ID NO:1 or SEQ ID NO:5;
wherein the presence of the rs67272382 insertion allele and/or the ss469415590 insertion allele indicates the individual is more likely to respond to a treatment for HCV infection than is an individual lacking either insertion allele.

In one embodiment, an individual having the rs67272382 insertion allele and/or the ss469415590 insertion allele is at least 1.5× (fold), 2.0×, 2.5×, 3.0×, 4.0×, or 5.0× more likely to respond to treatment for an HCV infection than is an individual lacking either insertion allele. In one embodiment, the likelihood that an individual having the rs67272382 insertion allele and/or the ss469415590 insertion allele will respond to treatment for an HCV infection has an odds ratio of at least about 1.2, at least about 1.4, at least about 1.6, at least about 1.8, at least about 2.0, at least about 2.2, at least about 2.4, at least about 2.6, at least about 2.8, at least about 3.0, at least about 3.2, at least about 3.4, at least about 2.6, at least about 3.8, at least about 4.0, at least about 4.2, at least about 4.4, at least about 4.6, at least about 4.8 or at least about 5.0. In one embodiment, the individual is predicted to be able to respond to a treatment for an HCV infection. In one embodiment, the treatment comprises using IFN-α, IFN-λ, pegylated-interferon, anti-protease drugs, anti-viral drugs, or combinations thereof.

One embodiment of the present invention is a method for predicting the likelihood of an individual to spontaneously clear an HCV infection, the method comprising:
a) obtaining a biological sample from the individual;
b) analyzing the sample to determine if the rs67272382 deletion allele is present at a locus in chromosome 19 represented by SEQ ID NO:3;
wherein the presence of the rs67272382 deletion allele indicates the individual is less likely to clear an HCV infection than is an individual lacking the rs67272382 deletion allele.

In one embodiment, an individual having the rs67272382 deletion allele is at least 1.5× (fold), 2.0×, 2.5×, 3.0×, 4.0×, or 5.0× less likely to spontaneously clear an HCV infection than is an individual lacking the rs67272382 deletion allele. In one embodiment, the likelihood that an individual having the rs67272382 deletion allele will fail to spontaneously clear an HCV infection has an odds ratio of at least about 1.2, at least about 1.4, at least about 1.6, at least about 1.8, at least about 2.0, at least about 2.2, at least about 2.4, at least about 2.6, at least about 2.8, at least about 3.0, at least about 3.2, at least about 3.4, at least about 2.6, at least about 3.8, at least about 4.0, at least about 4.2, at least about 4.4, at least about 4.6, at least about 4.8 or at least about 5.0. In one embodiment, the individual is predicted to be unable to spontaneously clear an HCV infection.

One embodiment of the present invention is a method for predicting the likelihood that an individual will respond to a treatment for HCV infection, the method comprising:
a) obtaining a biological sample from the individual;
b) analyzing the sample to determine if the rs67272382 deletion allele is present at a locus in chromosome 19 represented by SEQ ID NO:3;
wherein the presence of the rs67272382 deletion allele indicates the individual is less likely to respond to treatment for an HCV infection than is an individual lacking the rs67272382 deletion allele.
   In one embodiment, an individual having the rs67272382 deletion allele is at least 1.5× (fold), 2.0×, 2.5×, 3.0×, 4.0×, or 5.0× less likely to respond to treatment for an HCV infection than is an individual lacking the rs67272382 deletion allele (i.e., having the corresponding insertion alleles). In one embodiment, the likelihood that an individual having the rs67272382 deletion allele will fail to respond to treatment for an HCV infection has an odds ratio of at least about 1.2, at least about 1.4, at least about 1.6, at least about 1.8, at least about 2.0, at least about 2.2, at least about 2.4, at least about 2.6, at least about 2.8, at least about 3.0, at least about 3.2, at least about 3.4, at least about 2.6, at least about 3.8, at least about 4.0, at least about 4.2, at least about 4.4, at least about 4.6, at least about 4.8 or at least about 5.0. In one embodiment, the individual is predicted to be unable to respond to a treatment for an HCV infection. In one embodiment, the treatment comprises using IFN-α, IFN-λ, pegylated-interferon, anti-protease drugs, anti-viral drugs, or combinations thereof.

One embodiment of the present invention is a method for predicting the likelihood of an individual to spontaneously clear an HCV infection, the method comprising:
a) obtaining a biological sample from the individual;
b) analyzing the sample to determine if the ss469415590 deletion allele is present at a locus in chromosome 19 represented by SEQ ID NO:7;
wherein the presence of the ss469415590 deletion allele indicates the individual is less likely to clear an HCV infection than is an individual lacking the ss469415590 deletion allele.
   In one embodiment, an individual having the ss469415590 deletion allele is at least 1.5× (fold), 2.0×, 2.5×, 3.0×, 4.0×, or 5.0× less likely to spontaneously clear an HCV infection than is an individual lacking the ss469415590 deletion allele. In one embodiment, the likelihood that an individual having the ss469415590 deletion allele will fail to spontaneously clear an HCV infection has an odds ratio of at least about 1.2, at least about 1.4, at least about 1.6, at least about 1.8, at least about 2.0, at least about 2.2, at least about 2.4, at least about 2.6, at least about 2.8, at least about 3.0, at least about 3.2, at least about 3.4, at least about 2.6, at least about 3.8, at least about 4.0, at least about 4.2, at least about 4.4, at least about 4.6, at least about 4.8 or at least about 5.0. In one embodiment, the individual is predicted to be unable to spontaneously clear an HCV infection.

One embodiment of the present invention is a method for predicting the likelihood that an individual will respond to a treatment for HCV infection, the method comprising:
a) obtaining a biological sample from the individual;
b) analyzing the sample to determine if the ss469415590 deletion allele is present at a locus in chromosome 19 represented by SEQ ID NO:7;
wherein the presence of the ss469415590 deletion allele indicates the individual is less likely to respond to treatment for an HCV infection than is an individual lacking the ss469415590 deletion allele.
   In one embodiment, an individual having the ss469415590 deletion allele is at least 1.5× (fold), 2.0×, 2.5×, 3.0×, 4.0×, or 5.0× less likely to respond to treatment for an HCV infection than is an individual lacking the ss469415590 deletion allele (i.e., having the corresponding insertion alleles). In one embodiment, the likelihood that an individual having the ss469415590 deletion allele will fail to respond to treatment for an HCV infection has an odds ratio of at least about 1.2, at least about 1.4, at least about 1.6, at least about 1.8, at least about 2.0, at least about 2.2, at least about 2.4, at least about 2.6, at least about 2.8, at least about 3.0, at least about 3.2, at least about 3.4, at least about 2.6, at least about 3.8, at least about 4.0, at least about 4.2, at least about 4.4, at least about 4.6, at least about 4.8 or at least about 5.0. In one embodiment, the individual is predicted to be unable to respond to a treatment for an HCV infection. In one embodiment, the treatment comprises using IFN-α, IFN-λ, pegylated-interferon, anti-protease drugs, anti-viral drugs, or combinations thereof.

It will be appreciated by those skilled in the art that because mammals have pairs of chromosomes, they have two copies of the IL28B region in the genome, the sequences of which are not necessarily identical. That is, while the IL28B region on one chromosome may contain one allele of a polymorphism (e.g., rs67272382), the IL28B region of the other chromosome may contain the same or a different allele of that polymorphism. In instances where two loci in an individual contain different sequences (e.g. an allele and the wild-type sequence, two different alleles), the individual is referred to as being heterozygous for that loci. In instances where two loci in an individual contain the same sequence (e.g., both contain the same allele), the individual is referred to as being homozygous for that loci. The presence of one copy of a polymorphism can have a different effect on the likelihood of spontaneously clearing an HCV infection, or responding to an HCV treatment, than the presence of two copies of the same polymorphism. Thus, an individual who has two copies of an rs67272382 insertion allele or an ss469415590 insertion allele is more likely to spontaneously clear an HCV infection, or respond to a treatment for HCV infection, than is an individual who only has one copy of such an allele. Likewise, an individual who has one copy of an rs67272382 insertion allele, or an ss469415590 insertion allele, is more likely to spontaneously clear an HCV infection, or respond to treatment for HCV, than is an individual who does not carry the rs67272382 insertion allele, or the ss469415590 insertion allele.

Thus, one embodiment of the present invention is a method for predicting the likelihood that an individual will spontaneously clear an HCV infection, or respond to treatment for an HCV infection, the method comprising:
a) obtaining a biological sample from the individual; and
b) analyzing the sample to determine which allele of the rs67272382 polymorphism, or the ss469415590 polymorphism, is present on each chromosome at a locus in chromosome 19 represented by SEQ ID NO:1, SEQ ID NO:3, SEQ ID NO:5 or SEQ ID NO:7;
wherein if the individual is heterozygous for the insertion allele of the rs67272382, or the ss469415590, polymorphism, predicting that the individual is less likely to spontaneously clear an HCV infection, or respond to treatment for an HCV infection, than is an individual that is homozygous for the rs67272382, or the ss469415590, insertion allele, but more likely to spontaneously clear an HCV infection, or respond to treatment for an HCV infection, than is an individual homozygous for the rs67272382, or ss469415590, deletion allele.
   In one embodiment, an individual heterozygous for the rs67272382, or the ss469415590, insertion allele is at least 1.5× (fold), 2.0×, 2.5×, 3.0×, 4.0×, or 5.0× more likely to spontaneously clear an HCV infection, or respond to treatment for an HCV infection, than is an individual homozygous for the rs67272382, or the ss469415590, deletion allele. In one embodiment, an individual heterozygous for the rs67272382, or the ss469415590, insertion allele is at least 1.5× (fold), 2.0×, 2.5×, 3.0×, 4.0×, or 5.0× less likely to spontaneously clear an HCV infection, or respond to treatment for an HCV infection, than is an individual homozygous for the rs67272382, or the ss469415590, insertion allele. In one embodiment, the likelihood of an individual heterozygous for the rs67272382, or the ss469415590, insertion allele, being more likely to spontaneously clear an HCV infection, or respond to treatment for an HCV infection, than an individual homozygous for the rs67272382, or the ss469415590, deletion allele has an odds ratio of at least about 1.2, at least about 1.4, at least about 1.6, at least about 1.8, at least about 2.0, at least about 2.2, at least about 2.4, at least about 2.6, at least about 2.8, at least about 3.0, at least about 3.2, at least about 3.4, at least about 2.6, at least about 3.8, at least about 4.0, at least about 4.2, at least about 4.4, at least about 4.6, at least about 4.8 or at least about 5.0. In one embodiment, the likelihood of an individual heterozygous for the rs67272382, or the ss469415590, insertion allele, being less likely to spontaneously clear an HCV infection, or respond to treatment for an HCV infection, than an individual homozygous for the rs67272382, or the ss469415590, insertion allele has an odds ratio of at least about 1.2, at least about 1.4, at least about 1.6, at least about 1.8, at least about 2.0, at least about 2.2, at least about 2.4, at least about 2.6, at least about 2.8, at least about 3.0, at least about 3.2, at least about 3.4, at least about 2.6, at least about 3.8, at least about 4.0, at least about 4.2, at least about 4.4, at least about 4.6, at least about 4.8 or at least about 5.0. In one embodiment, an individual heterozygous for the rs67272382, or the ss469415590, insertion allele is predicted to be more likely to spontaneously clear an HCV infection, or respond to treatment for an HCV infection, than an individual homozygous for the rs67272382, or the ss469415590, deletion allele. In one embodiment, an individual heterozygous for the rs67272382, or the ss469415590, insertion allele is predicted to be less likely to spontaneously clear an HCV infection, or respond to treatment for an HCV infection, than an individual homozygous for the rs67272382, or the ss469415590, insertion allele. In one embodiment, the treatment comprises using IFN-α, IFN-λ, pegylated-interferon, anti-protease drugs, anti-viral drugs, or combinations thereof.

One embodiment of the present invention is a method for predicting the likelihood that an individual will spontaneously clear an HCV infection, or respond to treatment for an HCV infection, the method comprising:
a) obtaining a biological sample from the individual; and
b) analyzing the sample to determine which allele of the rs67272382 polymorphism, or the ss469415590 polymorphism, is present on each chromosome at a locus in chromosome 19 represented by SEQ ID NO:1, SEQ ID NO:3, SEQ ID NO:5 or SEQ ID NO:7;
wherein if the individual is homozygous for the insertion allele of the rs67272382, or the ss469415590, polymorphism, predicting that the individual is more likely to spontaneously clear an HCV infection, or respond to treatment for an HCV infection, than is an individual that is heterozygous for the rs67272382, or the ss469415590, insertion allele, and is also more likely to spontaneously clear an HCV infection, or respond to treatment for an HCV infection, than is an individual that is homozygous for the rs67272382, or the ss469415590, deletion allele.
   In one embodiment, an individual homozygous for the rs67272382, or the ss469415590, insertion allele is at least 1.5× (fold), 2.0×, 2.5×, 3.0×, 4.0×, or 5.0× more likely to spontaneously clear an HCV infection, or respond to treatment for an HCV infection, than is an individual heterozygous for the rs67272382, or the ss469415590, insertion allele. In one embodiment, an individual homozygous for the rs67272382, or the ss469415590, insertion allele is at least 1.5× (fold), 2.0×, 2.5×, 3.0×, 4.0×, or 5.0× more likely to spontaneously clear an HCV infection, or respond to treatment for an HCV infection, than is an individual homozygous for the rs67272382, or the ss469415590, deletion allele. In one embodiment, the likelihood of an individual homozygous for the rs67272382, or the ss469415590, insertion allele, being more likely to spontaneously clear an HCV infection, or respond to treatment for an HCV infection, than an individual heterozygous for the rs67272382, or the ss469415590, insertion allele has an odds ratio of at least about 1.2, at least about 1.4, at least about 1.6, at least about 1.8, at least about 2.0, at least about 2.2, at least about 2.4, at least about 2.6, at least about 2.8, at least about 3.0, at least about 3.2, at least about 3.4, at least about 2.6, at least about 3.8, at least about 4.0, at least about 4.2, at least about 4.4, at least about 4.6, at least about 4.8 or at least about 5.0. In one embodiment, the likelihood of an individual homozygous for the rs67272382, or the ss469415590, insertion allele, being more likely to spontaneously clear an HCV infection, or respond to treatment for an HCV infection, than an individual homozygous for the rs67272382, or the ss469415590, deletion allele has an odds ratio of at least about 1.2, at least about 1.4, at least about 1.6, at least about 1.8, at least about 2.0, at least about 2.2, at least about 2.4, at least about 2.6, at least about 2.8, at least about 3.0, at least about 3.2, at least about 3.4, at least about 2.6, at least about 3.8, at least about 4.0, at least about 4.2, at least about 4.4, at least about 4.6, at least about 4.8 or at least about 5.0. In one embodiment, an individual homozygous for the rs67272382, or the ss469415590, insertion allele is predicted to be more likely to spontaneously clear an HCV infection, or respond to treatment for an HCV infection, than is an individual heterozygous for the rs67272382, or the ss469415590, insertion allele. In one embodiment, an individual homozygous for the rs67272382, or the ss469415590, insertion allele is predicted to be more likely to spontaneously clear an HCV infection, or respond to treatment for an HCV infection, than an individual homozygous for the rs67272382, or the ss469415590, deletion allele. In one embodiment, the treatment comprises using IFN-α, IFN-λ, pegylated-interferon, anti-protease drugs, anti-viral drugs, or combinations thereof.

One embodiment of the present invention is a method for predicting the likelihood that an individual will spontaneously clear an HCV infection, or respond to treatment for an HCV infection, the method comprising:
a) obtaining a biological sample from the individual; and
b) analyzing the sample to determine which allele of the rs67272382, or the ss469415590, polymorphism is present on each chromosome at a locus in chromosome 19 represented by SEQ ID NO:1;
wherein if the individual is heterozygous for the deletion allele of rs67272382, or ss469415590, predicting that the individual is more likely to spontaneously clear an HCV infection, or respond to treatment for an HCV infection, than is an individual that is homozygous for the rs67272382, or the ss469415590, deletion allele, but less likely to spontaneously clear an HCV infection, or respond to treatment for an HCV infection, than an individual homozygous for the rs67272382, or the ss469415590, insertion allele.
   In one embodiment, an individual heterozygous for the rs67272382, or the ss469415590, deletion allele is at least 1.5× (fold), 2.0×, 2.5×, 3.0×, 4.0×, or 5.0× more likely to spontaneously clear an HCV infection or respond to treatment for an HCV infection, than is an individual homozygous for the rs67272382, or the ss469415590, deletion allele. In one embodiment, an individual heterozygous for the rs67272382, or the ss469415590, deletion allele is at least 1.5× (fold), 2.0×, 2.5×, 3.0×, 4.0×, or 5.0× less likely to spontaneously clear an HCV infection, or respond to treatment for an HCV infection, than is an individual homozygous for the rs67272382, of the ss469415590, insertion allele. In one embodiment, the likelihood of an individual heterozygous for the rs67272382, or the ss469415590, deletion allele, being more likely to spontaneously clear an HCV infection, or respond to treatment for an HCV infection, than an individual homozygous for the rs67272382, or the ss469415590, deletion allele has an odds ratio of at least about 1.2, at least about 1.4, at least about 1.6, at least about 1.8, at least about 2.0, at least about 2.2, at least about 2.4, at least about 2.6, at least about 2.8, at least about 3.0, at least about 3.2, at least about 3.4, at least about 2.6, at least about 3.8, at least about 4.0, at least about 4.2, at least about 4.4, at least about 4.6, at least about 4.8 or at least about 5.0. In one embodiment, the likelihood of an individual heterozygous for the rs67272382, or the ss469415590, deletion allele, being less likely to spontaneously clear an HCV infection, or respond to treatment for an HCV infection, than an individual homozygous for the rs67272382, or the ss469415590, insertion allele has an odds ratio of at least about 1.2, at least about 1.4, at least about 1.6, at least about 1.8, at least about 2.0, at least about 2.2, at least about 2.4, at least about 2.6, at least about 2.8, at least about 3.0, at least about 3.2, at least about 3.4, at least about 2.6, at least about 3.8, at least about 4.0, at least about 4.2, at least about 4.4, at least about 4.6, at least about 4.8 or at least about 5.0. In one embodiment, an individual heterozygous for the rs67272382, or the ss469415590, deletion allele is predicted to be more likely to spontaneously clear an HCV infection, or respond to treatment for an HCV infection, than an individual homozygous for the rs67272382, or the ss469415590, deletion allele. In one embodiment, an individual heterozygous for the rs67272382, or the ss469415590, deletion allele is predicted to be less likely to spontaneously clear an HCV infection, or respond to treatment for an HCV infection, than an individual homozygous for the rs67272382, or the ss469415590, insertion allele. In one embodiment, the treatment comprises using IFN-α, IFN-λ, pegylated-interferon, anti-protease drugs, anti-viral drugs, or combinations thereof.

One embodiment of the present invention is a method for predicting the likelihood that an individual will spontaneously clear an HCV infection, or respond to treatment for an HCV infection, the method comprising:
a) obtaining a biological sample from the individual; and
b) analyzing the sample to determine which allele of the rs67272382, or the ss469415590, polymorphism is present on each chromosome at a locus in chromosome 19 represented by SEQ ID NO:1;
wherein if the individual is homozygous for the deletion allele of the rs67272382, or the ss469415590, polymorphism, predicting that the individual is less likely to spontaneously clear an HCV infection, or respond to treatment for an HCV infection, than is an individual that is heterozygous for the rs67272382, or the ss469415590, deletion allele or homozygous for the rs67272382, or the ss469415590, insertion allele.
   In one embodiment, an individual homozygous for the rs67272382, or the ss469415590, deletion allele is at least 1.5× (fold), 2.0×, 2.5×, 3.0×, 4.0×, or 5.0× less likely to spontaneously clear an HCV infection, or respond to treatment for an HCV infection, than is an individual heterozygous for the rs67272382, or the ss469415590, deletion allele. In one embodiment, an individual homozygous for the rs67272382, or the ss469415590, deletion allele is at least 1.5× (fold), 2.0×, 2.5×, 3.0×, 4.0×, or 5.0× less likely to spontaneously clear an HCV infection, or respond to treatment for an HCV infection, than is an individual homozygous for the rs67272382, or the ss469415590, insertion allele. In one embodiment, the likelihood of an individual homozygous for the rs67272382, or the ss469415590, deletion allele, being less likely to spontaneously clear an HCV infection, or respond to treatment for an HCV infection, than an individual heterozygous for the rs67272382, or the ss469415590, deletion allele has an odds ratio of at least about 1.2, at least about 1.4, at least about 1.6, at least about 1.8, at least about 2.0, at least about 2.2, at least about 2.4, at least about 2.6, at least about 2.8, at least about 3.0, at least about 3.2, at least about 3.4, at least about 2.6, at least about 3.8, at least about 4.0, at least about 4.2, at least about 4.4, at least about 4.6, at least about 4.8 or at least about 5.0. In one embodiment, the likelihood of an individual homozygous for the rs67272382, or the ss469415590, deletion allele, being less likely to spontaneously clear an HCV infection, or respond to treatment for an HCV infection, than an individual homozygous for the rs67272382, or the ss469415590, insertion allele has an odds ratio of at least about 1.2, at least about 1.4, at least about 1.6, at least about 1.8, at least about 2.0, at least about 2.2, at least about 2.4, at least about 2.6, at least about 2.8, at least about 3.0, at least about 3.2, at least about 3.4, at least about 2.6, at least about 3.8, at least about 4.0, at least about 4.2, at least about 4.4, at least about 4.6, at least about 4.8 or at least about 5.0. In one embodiment, an individual homozygous for the rs67272382, or the ss469415590, deletion allele is predicted to be less likely to spontaneously clear an HCV infection, or respond to treatment for an HCV infection, than is an individual heterozygous for the rs67272382, or the ss469415590, deletion allele. In one embodiment, an individual homozygous for the rs67272382, or the ss469415590, deletion allele is predicted to be less likely to spontaneously clear an HCV infection, or respond to treatment for an HCV infection, than an individual homozygous for the rs67272382, or the ss469415590, insertion allele. In one embodiment, the treatment comprises using IFN-α, IFN-λ, pegylated-interferon, anti-protease drugs, anti-viral drugs, or combinations thereof.

It should be noted that while determining which allele is present at a specific locus can involve determining the sequence of several nucleotides at that locus, it can also involve detecting the specific nucleotide changes that make up the alleles of a polymorphism. Thus, for example, the presence of specific alleles of the rs67272382 polymorphism can be determined by detecting the presence, or absence, of a thymidine at a location corresponding to position 27 and/or 28 of SEQ ID NO:1 or SEQ ID NO:5, at the locus represented by SEQ ID NO:1 or SEQ ID NO:5. Likewise, the presence of specific alleles of the ss469415590 polymorphism can be determined by detecting the presence, or absence, of a thymidine at a position corresponding to position 27 of SEQ ID NO:1, and/or a guanine at a position corresponding to position 28 of SEQ ID NO:1 or SEQ ID NO:5, at the locus represented by SEQ ID NO:1 or SEQ ID NO:5.

It is well known in the art that chromosomes are composed of double stranded DNA molecules. Thus, while the present invention refers to detecting the presence of particular nucleotides in a particular strand or sequence (e.g., SEQ ID NO:1, SEQ ID NO:3, SEQ ID NO:5, SEQ ID NO:7, etc.), the invention may also be practiced by detecting the corresponding nucleotide in the complementary strand. For example, while the presence of the rs67272382 insertion allele may be determined by detecting a thymidine residue at a location on chromosome 19 corresponding to position 27 of SEQ ID NO:1, at the locus represented by SEQ ID NO:1 or SEQ ID NO:5, it can also be determined by detecting an adenine residue at the complementary nucleotide position on the opposite DNA strand of chromosome 19. The locus complementary to the locus represented by SEQ ID NO:1 is represented by SEQ NO:2. Thus, the presence of the rs67272382 insertion allele may be determined by detecting an adenine at a location on chromosome 19 corresponding to position 26 of SEQ ID NO:2. Similarly, the presence of the ss469415590 insertion allele may be determined by detecting a thymidine residue at a location on chromosome 19 corresponding to position 27 of SEQ ID NO:5, at the locus represented by SEQ ID NO:5. However, the presence of the ss469415590 insertion allele may be determined by detecting an adenine at the complementary nucleotide position on the opposite DNA strand of chromosome 19. The locus complementary to the locus represented by SEQ ID NO:5 is represented by SEQ ID NO:6. Thus, the presence of the ss469415590 insertion allele may be determined by detecting an adenine at a location on chromosome 19 corresponding to position 26 of SEQ ID NO:2. Table 1 below lists the sequences of the alleles of the polymorphisms referred to herein, along with their complementary sequences.

TABLE 1

| SEQ ID | | |
|---|---|---|
| NO: | Organism | Description |
| 1 | Human | rs67272382-insertion allele; |
| | | GCGAGAGCAGGCAGCGCCGGGGGGCC[T]TCTGCGATC |
| | | ACCGTGCACAGGACCC |
| 2 | | Complement of SEQ ID NO: 1 |
| 3 | Human | rs67272382 alleles T/− −deletion allele |
| | | GCGAGAGCAGGCAGCGCCGGGGGGCC[-] |
| | | TCTGCGATCACCGTGCACAGGACCC |
| 4 | | Complement of SEQ ID NO: 3 |
| 5 | Human | ss469415590-insertion allele |
| | | GCGAGAGCAGGCAGCGCCGGGGGGCC[TT]CTGCGATC |
| | | ACCGTGCACAGGACCC |
| 6 | | Complement of SEQ ID NO: 5 |
| 7 | Human | ss469415590, alleles TT/−G-deletion allele; |
| | | ΔGGCGAGAGCAGGCAGCGCCGGGGGGCC[−G] |
| | | CTGCGATCACCGTGCACAGGACCC |
| 8 | | Complement of SEQ ID NO: 7 |
| 9 | Synthetic | ss469415590_F Forward primer |
| | | GCCTGCTGCAGAAGCAGAGAT |
| 10 | Synthetic | ss469415590_R Reverse primer |
| | | GCTCCAGCGAGCGGTAGTG |
| 11 | Synthetic | ss469415590_VIC (TT non-risk allele) |
| | | ATCGCAGAAGGCC |
| 12 | Synthetic | ss469415590_FAM (deltaG risk allele) |
| | | ATCGCAGCGGCCC |
| 13 | Synthetic | Forward primer for Sanger sequencing |
| | | CGAACCAGGGTTGAATTGC |
| 14 | Synthetic | Reverse primer for Sanger sequencing |
| | | GCACTGCAGACAGGAGTGAG |
| 15 | Human | Sequence amplified by SEQ ID NO's 5 and 6 |
| | | and used for TaqMan genotyping |
| | | GCCTGCTGCAGAAGCAGAGATGCGGCCGAGTGTCTGG |
| | | GCCGCAGTGGCCGC[G/c]GGGCTGTGGGTCCTGT[G/a]C |
| | | ACGGTGATCGCAG[AA/−C]GGCCCCCCGGCGCTGCCT |
| | | GCTCTC[G/c]CACTACCGCTCGCTGGAGC |
| 16 | | Complement of SEQ ID NO: 15 |
| 17 | Human | rs74597329, allele 1 |
| | | CGAGAGCAGGCAGCGCCGGGGGGCCT[T]CTGCGATCA |
| | | CCGTGCACAGGACCCA |
| 18 | | Complement of SEQ ID NO: 17 |
| 19 | Human | rs74597329, allele 2 |
| | | CGAGAGCAGGCAGCGCCGGGGGGCCT[G]CTGCGATCA |
| | | CCGTGCACAGGACCCA |
| 20 | | Complement of SEQ ID NO: 19 |
| 21 | Human | rs117648444, allele 1 |
| | | CAACTGCTCCTTCCGCCCCAGGAGGGAT[C]CTCCGCGG |
| | | CCATCCGTGAGGCCCG |
| 22 | | Complement of SEQ ID NO: 21 |
| 23 | Human | rs117648444, allele 2 |
| | | CAACTGCTCCTTCCGCCCCAGGAGGGAT[T]CTCCGCGG |
| | | CCATCCGTGAGGCCCG |
| 24 | | Complement of SEQ ID NO: 23 |
| 25 | Human | ss539198934, allele 1 |
| | | CACTCTGGGCCTTGCCTCTGACCCCGCCCCCTC[T]GGCAGCA |
| | | CGGAAACCTCCACGCCATTGGCTGCCGAAAGCAGCTCC |
| 26 | | Complement of SEQ ID NO: 25 |
| 27 | Human | ss539198934, allele 2 |
| | | CACTCTGGGCCTTGCCTCTGACCCCGCCCCCTC[C]GGCAGCA |
| | | CGGAAACCTCCACGCCATTGGCTGCCGAAAGCAGCTCC |
| 28 | | Complement of SEQ ID NO: 27 |
| 29 | Human | rs73555604, allele 1 |
| | | GGGCTGTGGGTCCTGT[G]CACGGTGATCGCAGC |
| 30 | | Complement of SEQ ID NO: 29 |
| 31 | Human | rs73555604, allele 2 |
| | | GGGCTGTGGGTCCTGT[A]CACGGTGATCGCAGC |
| 32 | | Complement of SEQ ID NO: 31 |
| 33 | Human | rs137902769, allele 1 |
| | | CTGGCTGCACACTC[T]GGGCCTTGCCTCTGA |
| 34 | | Complement of SEQ ID NO: 33 |
| 35 | Human | rs137902769, allele 2 |
| | | CGGCTGGCTGCACACTC[A]GGGCCTTGCCTCTGA |
| 36 | | Complement of SEQ ID NO: 35 |
| 37 | Human | rs142981501, allele 1 |
| | | GAGGAAGAGGCGCTGAGCTGGGGGCAGC[G]CAACTGC |
| | | TCCTTCCGCCCCAGGAGGGAT |
| 38 | | Complement of SEQ ID NO: 37 |
| 39 | Human | rs142981501, allele 2 |
| | | GAGGAAGAGGCGCTGAGCTGGGGGCAGC[C]CAACTGC |
| | | TCCTTCCGCCCCAGGAGGGAT |
| 40 | | Complement of SEQ ID NO: 39 |
| 41 | Synthetic | rs12979860_F-forward primer |
| | | GCCTGTCGTGTACTGAACCA |
| 42 | Synthetic | rs12979860_r-reverse primer |
| | | GCGCGGAGTGCAATTCAAC |
| 43 | Synthetic | rs12979860_FAM (T risk allele) |
| | | CTGGTTCACGCCTTC |
| 44 | Synthetic | rs12979860_VIC (C non-risk allele) |
| | | TGGTTCGCGCCTTC |

Based on the teaching herein, one skilled in the art would understand how to determine which base is present at a specific location using either strand of DNA in chromosome 19.

It will be understood by those skilled in the art that chromosomes consist of extremely large polynucleotide sequences, consisting of millions of nucleotides bases. It is also clear that polymorphisms of the present invention consist of just a few nucleotides out of the millions of nucleotides in the genome. Thus, it is essential that the genomic location of such nucleotides be clearly understood. According to the present invention, the nucleotide variations being detected are located on chromosome 19, upstream of the IL28B gene. In one embodiment, the nucleotide variations being detected are located approximately 3500 nucleotides upstream of the IL28B gene translation start site. As used herein, approximately, about, and the like, in the context of distance along a chromosome (e.g., number of nucleotides from a specific location) refer to a variation of no more than 250, preferably 100, preferably 50, preferably 25, preferably 10, and more preferably 5 nucleotides. Thus, for example, approximately 3500 nucleotides can be a range of 3,750 nucleotides to 3,250 nucleotides.

In one embodiment, the nucleotide variations being detected are located about 3,574 nucleotides upstream of the translation start site for the IL28B gene. In one embodiment, the nucleotide variations being detected are located at about position 39,739,154 base pairs (bp) on chromosome 19, all coordinates based on February 2009 human genome reference (GRch37/hg19). In one embodiment, the nucleotide variations being detected are located between positions 39,739,152 bp and position 39,739,157 bp, more preferably between positions 39,739,153 bp and position 39,739,156 bp on chromosome 19, all coordinates based on February 2009 human genome reference (GRch37/hg19). In one embodiment, the nucleotide variations being detected are located at position 39,739,154 bp and position 39,739,155 bp on chromosome 19, all coordinates based on February 2009 human genome reference (GRch37/hg19).

In humans, the IL28B locus generally refers to a genomic DNA region located within the long arm of chromosome 19 encoding IL28B (which belongs to the IFNλ family). The IL-28B (IFNλ3) gene has 5 protein-coding exons, and encodes a 20 kDa secreted monomeric protein. It has recently been reported that the IL28B cytokine could be an interesting substitute to IFN-α for the treatment of HCV-infected patients who are, or who become, resistant to IFN-α ([38]). It should be understood that, for reasons of clarity, while it is convenient to refer to polymorphisms of the present invention (i.e., rs67272382/, and ss469415590) as being located upstream of the IL28B gene, it should be understood that such polymorphisms are not located within the IL28B gene.

It will be clear to those skilled in the art that listing the sequence of an entire chromosome herein is impractical. Consequently, reference to polymorphisms in a database is made using a short, representative sequence from the region of the chromosome containing the polymorphism. Such representative sequences are referred to as Reference SNPs and are initially designated using the prefix “ss” (e.g., ss469415590). Upon release of an updated build of the relevant genome, the polymorphism will receive the prefix “rs”. It will be appreciated by those skilled in the art that while such sequences are referred to as reference single nucleotide polymorphisms, as has been described such polymorphisms may involve more than one nucleotide. According to the present invention, such polymorphisms may involve one, two three, four or more nucleotides. Nucleotide positions involved in making up a polymorphism may be adjacent to one another, or they may be within 25, preferably 20, and more preferably within 10 nucleotide positions of one another. In one embodiment of the present invention, the polymorphic marker is made up of two adjacent nucleotide positions.

In a preferred embodiment, a polymorphism of the present invention is located in a nucleic acid segment of the genome represented by SEQ ID NO:1, SEQ ID NO:2, SEQ ID NO:3, SEQ ID NO:4, SEQ ID NO:5, SEQ ID NO:6 or SEQ ID NO:7. In one embodiment, the polymorphism is a deletion of the nucleotide corresponding to the nucleotide at position 27 of SEQ ID NO:1 or SEQ ID NO:5. In one embodiment, the polymorphism is a substitution of the thymidine corresponding to position 28 of SEQ ID NO; 1 or SEQ ID NO:5 with guanine, adenine or cytosine. In one embodiment, the polymorphism is a deletion of the nucleotide corresponding to the nucleotide at position 27 of SEQ ID NO:1 or SEQ ID NO:5 and a substitution of the thymidine corresponding to position 28 of SEQ ID NO:1 or SEQ ID NO:5 with guanine, adenine or cytosine, such polymorphism being referred to as ss469415590. In one embodiment the polymorphism is rs67272382 (represented by SEQ ID NOs 1-4). In one embodiment, the polymorphism is ss469415590 which consists of a deletion of the nucleotide corresponding to the nucleotide at position 27 of SEQ ID NO:1 or SEQ ID NO:5 and a substitution of the thymidine corresponding to position 28 of SEQ ID NO:1 or SEQ ID NO:5 with guanine, adenine or cytosine. The ss469415590 polymorphism is represented by SEQ ID NOs 5-8).

One embodiment of the present invention is a method for predicting the likelihood of an individual to spontaneously clear an HCV infection, the method comprising:
a) obtaining a biological sample from the patient;
b) analyzing the sample in order to determine the presence or absence of particular nucleotides in a locus of chromosome 19 represented by SEQ ID NO:1 or SEQ ID NO:5, wherein the presence or absence of particular nucleotides at positions corresponding to positions 27 and 28 of SEQ ID NO:1 or SEQ ID NO:5 indicate the likelihood the individual will spontaneously clear an HCV infection.

In one embodiment, the presence of a thymidine at positions in chromosome 19 corresponding to positions 27 and 28 of SEQ ID NO:1 or SEQ ID NO:5, indicates the presence of the rs67272382 insertion allele or the ss469415590 insertion allele. Thus the individual is likely to spontaneously clear an HCV infection. In one embodiment, the absence of a thymidine at a position in chromosome 19 corresponding to positions 27 of SEQ ID NO:1 or SEQ ID NO:5, indicates the presence of the rs67272382 deletion allele or the ss469415590 deletion allele. An individual having such a deletion allele is less likely to spontaneously clear an HCV infection than is an individual having a thymidine at a position in chromosome 19 corresponding to positions 27 of SEQ ID NO:1 or SEQ ID NO:5 and a thymidine at a position in chromosome 19 corresponding to positions 28 of SEQ ID NO:1 or SEQ ID NO:5. Likewise, in one embodiment, the presence of guanidine at a position in chromosome 19 corresponding to position 28 of SEQ ID NO:1 or SEQ ID NO:5, indicates the presence of the ss469415590 deletion allele. In one embodiment, the absence of a thymidine at a position in chromosome 19 corresponding to position 27 of SEQ ID NO: 1 or SEQ ID NO:5, and the presence of guanidine at a position in chromosome 19 corresponding to position 28 of SEQ ID NO:1 or SEQ ID NO:5, indicates the presence of the ss469415590 deletion allele. An individual having such a deletion allele is less likely to spontaneously clear an HCV infection than is an individual having a thymidine that position.

One embodiment of the present invention is a method for predicting the likelihood of an individual responding to treatment for an HCV infection, the method comprising:
a) obtaining a biological sample from the patient;
b) analyzing the sample in order to determine the presence or absence of particular nucleotides in a locus of chromosome 19 represented by SEQ ID NO:1 or SEQ ID NO:5, wherein the presence or absence of particular nucleotides at positions corresponding to positions 27 and 28 of SEQ ID NO:1 or SEQ ID NO:5 indicate the likelihood the individual will spontaneously clear an HCV infection.

In one embodiment, the presence of a thymidine at positions in chromosome 19 corresponding to positions 27 and 28 of SEQ ID NO:1 or SEQ ID NO:5, indicates the presence of the rs67272382 insertion allele or the ss469415590 insertion allele. Thus the individual is likely to respond to treatment for an HCV infection. In one embodiment, the absence of a thymidine at a position in chromosome 19 corresponding to positions 27 of SEQ ID NO:1 or SEQ ID NO:5, indicates the presence of the rs67272382 deletion allele or the ss469415590 deletion allele. An individual having such a deletion allele is less likely to respond to treatment for an HCV infection than is an individual having a thymidine at a position in chromosome 19 corresponding to positions 27 of SEQ ID NO:1 or SEQ ID NO:5 and a thymidine at a position in chromosome 19 corresponding to positions 28 of SEQ ID NO:1 or SEQ ID NO:5. Likewise, in one embodiment, the presence of guanidine at a position in chromosome 19 corresponding to position 28 of SEQ ID NO:1 or SEQ ID NO:5, indicates the presence of the ss469415590 deletion allele. In one embodiment, the absence of a thymidine at a position in chromosome 19 corresponding to position 27 of SEQ ID NO: 1 or SEQ ID NO:5, and the presence of guanidine at a position in chromosome 19 corresponding to position 28 of SEQ ID NO:1 or SEQ ID NO:5, indicates the presence of the ss469415590 deletion allele. An individual having such a deletion allele is less likely to respond to treatment for an HCV infection than is an individual having a thymidine that position.

The disclosure thus far has focused on using the presence or absence of specific alleles of the rs67272382 and ss469415590 polymorphisms for determining the likelihood of an individual to spontaneously clear an HCV infection, or respond to treatment for an HCV infection. However, those skilled in the art will appreciate that phenotypic activity (e.g., spontaneously clearing an HCV infection) is often modulated by multiple genetic components. For example, the expression of a gene may be modulated by a protein expressed by a second gene, or by other genetic elements, such as enhancer elements. Moreover, different individuals may carry slightly different elements (i.e., different alleles), and these different elements may be identified using polymorphisms linked to the individual elements. Accordingly, in addition to determining which allele(s) of the rs67272382 and/or ss469415590 polymorphisms are present in an individual, methods of the present invention may comprise determining which alleles of various other polymorphisms are present. Thus, one embodiment of the present invention is a method for predicting the likelihood of an individual to spontaneously clear an HCV infection, the method comprising:
a) obtaining a biological sample from an individual;
b) analyzing the sample to identify which allele(s) of the rs67272382, or the ss469415590, polymorphism is/are present at a locus in chromosome 19 represented by SEQ ID NO:1; and
c) analyzing the sample to identify which allele(s) of at least one polymorphism selected from group consisting of rs74597329, rs117648444, ss539198934, rs73555604, rs137902769 and rs142981501, are present at the appropriate location of the individual's genome;
d) predicting the likelihood the individual will spontaneously clear an HCV infection based on the combination of alleles present in the individual.

In one embodiment, the presence of an rs67272382 deletion allele indicates that the individual is predicted to be less likely to spontaneously clear an HCV infection than is an individual lacking the rs67272382 deletion allele. In one embodiment, the presence of an ss469415590 deletion allele indicates the individual is less likely to spontaneously clear an HCV infection than is an individual lacking the ss469415590 deletion allele. In one embodiment, the presence of a particular allele of the rs74597329 polymorphism increases the likelihood that the individual will spontaneously clear an HCV infection. In one embodiment, the presence of an rs67272382, or an ss469415590 deletion allele, and the presence of a particular allele of the rs74597329 polymorphism indicates that the individual is less likely to spontaneously clear an HCV infection than is an individual lacking the rs67272382, or the ss469415590 deletion allele, but more likely to spontaneously clear an HCV infection than is a individual lacking the specific rs74597329 allele. In one embodiment, the presence of the “T” allele of the rs117648444 polymorphism (represented by SEQ ID NO:23) increases the likelihood that the individual will spontaneously clear an HCV infection. In one embodiment, the presence of an rs67272382, or an ss469415590 deletion allele, and the presence of the “T” allele of the rs117648444 polymorphism indicates that the individual is less likely to spontaneously clear an HCV infection than is an individual lacking the rs67272382, or the ss469415590 deletion allele, but more likely to spontaneously clear an HCV infection than is a individual lacking the rs117648444 “T” allele. In one embodiment, the presence of the “C” allele of the ss539198934 polymorphism (represented by SEQ ID NO:27) increases the likelihood that the individual will spontaneously clear an HCV infection. In one embodiment, the presence of an rs67272382, or an ss469415590 deletion allele, and the presence of the “C” allele of the ss539198934 polymorphism indicates that the individual is less likely to spontaneously clear an HCV infection than is an individual lacking the rs67272382, or the ss469415590 deletion allele, but more likely to spontaneously clear an HCV infection than is a individual lacking the ss539198934 “C” allele. In one embodiment, the presence of the “A” allele of the rs73555604 polymorphism (represented by SEQ ID NO:31) increases the likelihood that the individual will spontaneously clear an HCV infection. In one embodiment, the presence of an rs67272382, or an ss469415590 deletion allele, and the presence of the “A” allele of the rs73555604 polymorphism indicates that the individual is less likely to spontaneously clear an HCV infection than is an individual lacking the rs67272382, or the ss469415590 deletion allele, but more likely to spontaneously clear an HCV infection than is a individual lacking the rs73555604 “A” allele. In one embodiment, the presence of the “A” allele of the rs137902769 polymorphism (represented by SEQ ID NO:35) increases the likelihood that the individual will spontaneously clear an HCV infection. In one embodiment, the presence of an rs67272382, or an ss469415590 deletion allele, and the presence of the “A” allele of the rs137902769 polymorphism indicates that the individual is less likely to spontaneously clear an HCV infection than is an individual lacking the rs67272382, or the ss469415590 deletion allele, but more likely to spontaneously clear an HCV infection than is a individual lacking the rs137902769 “A” allele. In one embodiment, the presence of the “C” allele of the rs142981501 polymorphism (represented by SEQ IDNO:39) increases the likelihood that the individual will spontaneously clear an HCV infection. In one embodiment, the presence of an rs67272382, or an ss469415590 deletion allele, and the presence of the “C” allele of the rs142981501 polymorphism indicates that the individual is less likely to spontaneously clear an HCV infection than is an individual lacking the rs67272382, or the ss469415590 deletion allele, but more likely to spontaneously clear an HCV infection than is a individual lacking the rs142981501 “C” allele.

One embodiment of the present invention is a method for predicting the likelihood of an individual to respond to a treatment for an HCV infection, the method comprising:
a) obtaining a biological sample from the individual;
b) analyzing the sample to identify which allele(s) of the rs67272382, or the ss469415590, polymorphism is/are present at a locus in chromosome 19 represented by SEQ ID NO:1; and
c) analyzing the sample to identify which allele(s) of at least one polymorphism selected from group consisting of rs74597329, rs117648444, ss539198934, rs73555604, rs137902769 and rs142981501, are present at the appropriate location of the individuals genome;
d) predicting the likelihood the individual will respond to a treatment for an HCV infection based on the combination of alleles present in the individual.

In one embodiment, the presence of an rs67272382 deletion allele indicates that the individual is predicted to be less likely to respond to a treatment for an HCV infection than is an individual lacking the rs67272382 deletion allele. In one embodiment, the presence of an ss469415590 deletion allele indicates the individual is less likely to respond to a treatment for an HCV infection than is an individual lacking the ss469415590 deletion allele. In one embodiment, the presence of a particular allele of the rs74597329 polymorphism increases the likelihood that the individual will respond to a treatment for an HCV infection. In one embodiment, the presence of an rs67272382, or an ss469415590 deletion allele, and the presence of a particular allele of the rs74597329 polymorphism indicates that the individual is less likely to respond to a treatment for an HCV infection than is an individual lacking the rs67272382, or the ss469415590 deletion allele, but more likely to respond to a treatment for an HCV infection than is a individual lacking the specific rs74597329 allele. In one embodiment, the presence of the “T” allele of the rs117648444 polymorphism (represented by SEQ ID NO:23) increases the likelihood that the individual will respond to a treatment for an HCV infection. In one embodiment, the presence of an rs67272382, or an ss469415590 deletion allele, and the presence of the “T” allele of the rs117648444 polymorphism indicates that the individual is less likely to respond to a treatment for an HCV infection than is an individual lacking the rs67272382, or the ss469415590 deletion allele, but more likely to respond to a treatment for an HCV infection than is a individual lacking the rs117648444 “T” allele. In one embodiment, the presence of the “C” allele of the ss539198934 polymorphism (represented by SEQ ID NO:27) increases the likelihood that the individual will respond to a treatment for an HCV infection. In one embodiment, the presence of an rs67272382, or an ss469415590 deletion allele, and the presence of the “C” allele of the ss539198934 polymorphism indicates that the individual is less likely to respond to a treatment for an HCV infection than is an individual lacking the rs67272382, or the ss469415590 deletion allele, but more likely to respond to a treatment for an HCV infection than is a individual lacking the ss539198934 “C” allele. In one embodiment, the presence of the “A” allele of the rs73555604 polymorphism (represented by SEQ ID NO:31) increases the likelihood that the individual will respond to a treatment for an HCV infection. In one embodiment, the presence of an rs67272382, or an ss469415590 deletion allele, and the presence of the “A” allele of the rs73555604 polymorphism indicates that the individual is less likely to respond to a treatment for an HCV infection than is an individual lacking the rs67272382, or the ss469415590 deletion allele, but more likely to respond to a treatment for an HCV infection than is a individual lacking the rs73555604 “A” allele. In one embodiment, the presence of the “A” allele of the rs137902769 polymorphism (represented by SEQ ID NO:35) increases the likelihood that the individual will respond to a treatment for an HCV infection. In one embodiment, the presence of an rs67272382, or an ss469415590 deletion allele, and the presence of the “A” allele of the rs137902769 polymorphism indicates that the individual is less likely to respond to a treatment for an HCV infection than is an individual lacking the rs67272382, or the ss469415590 deletion allele, but more likely to respond to a treatment for an HCV infection than is a individual lacking the rs137902769 “A” allele. In one embodiment, the presence of the “C” allele of the rs142981501 polymorphism (represented by SEQ IDNO:39) increases the likelihood that the individual will respond to a treatment for an HCV infection. In one embodiment, the presence of an rs67272382, or an ss469415590 deletion allele, and the presence of the “C” allele of the rs142981501 polymorphism indicates that the individual is less likely to respond to a treatment for an HCV infection than is an individual lacking the rs67272382, or the ss469415590 deletion allele, but more likely to respond to a treatment for an HCV infection than is a individual lacking the rs142981501 “C” allele. In one embodiment, the treatment comprises administering IFN-α, IFN-λ, pegylated-interferon, anti-protease drugs, anti-viral drugs, or combinations thereof.

In one embodiment, the steps of analyzing the sample for the various polymorphism alleles are conducted in parallel. That is, an assay is used that determines which alleles are present in the sample, all at the same time. Alternatively, the sample is tested to determine which allele(s) of the rs67272382, and/or the ss469415590, polymorphisms is/are present, and only if a rs67272382, and/or a ss469415590, deletion allele is/are detected is the sample then tested to determine which alleles of at least one polymorphism selected from group consisting of rs74597329, rs117648444, ss539198934, rs73555604, rs137902769 and rs142981501 are present.

Since methods of the present invention can be used to predict an individual's response to a therapeutic treatment, such methods can be incorporated into a treatment plan. Thus, one embodiment of the present invention is a method of treating a patient suffering from a hepatitis C virus infection, the method comprising:
a) obtaining a biological sample from the individual;
b) analyzing the sample to identify which allele(s) of at least one polymorphism selected from the group consisting of rs67272382 and ss469415590 is/are present at a locus in chromosome 19 represented by SEQ ID NO:1, SEQ ID NO:3, SEQ ID NO:5 or SEQ ID NO:7; and
c) determining whether or not to administer treatment based on which allele is present.
   In one embodiment, the patient has an acute HCV infection. In one embodiment, the patient has a chronic HCV infection. In one embodiment, the presence of an rs67272382 insertion allele indicates the individual is likely to respond to a treatment for an HCV infection and thus a treatment is administered. In one embodiment, the presence of an ss469415590 insertion allele indicates the individual is likely to respond to a treatment for an HCV infection and thus a treatment is administered. In one embodiment, the treatment comprises using IFN-α, IFN-λ, pegylated-interferon, anti-protease drugs, anti-viral drugs, or combinations thereof.

In one embodiment, the presence of a rs67272382 deletion allele indicates that the individual is unlikely to respond to administration of a treatment for an HCV infection and treatment is not administered. In one embodiment, the presence of a ss469415590 deletion allele indicates that the individual is unlikely to respond to administration of a treatment for an HCV infection and treatment is not administered.

One embodiment of the present invention is a method of treating a patient suffering from a hepatitis C virus infection, the method comprising:
a) obtaining a biological sample from the individual;
b) analyzing the sample to identify which allele(s) of at least one polymorphism selected from the group consisting of rs67272382 and ss469415590 is/are present at a locus in chromosome 19 represented by SEQ ID NO:1, SEQ ID NO:3, SEQ ID NO:5 or SEQ ID NO:7;
c) treating the individual if at least one rs67272382 insertion allele, or at least one ss469415590 insertion allele, is identified in the individual, wherein the treatment comprises administering at least one pharmaceutical composition.
   In one embodiment, the composition comprises IFN-α, IFN-λ, pegylated-interferon, anti-protease drugs, anti-viral drugs, or combinations thereof.

One embodiment of the present invention is a method of treating a patient suffering from a hepatitis C virus infection, the method comprising:
a) obtaining a biological sample from the individual;
b) analyzing the sample to identify which allele(s) of at least one polymorphism selected from the group consisting of rs67272382 and ss469415590 is/are present at a locus in chromosome 19 represented by SEQ ID NO:1;
c) treating the individual if at least one rs67272382 deletion allele, or at least one ss469415590 deletion allele, is identified in the individual, wherein treatment comprises withholding administration of at least one anti-viral composition.

In one embodiment, the composition comprises IFN-α, IFN-λ, pegylated-interferon, anti-protease drugs, anti-viral drugs, or combinations thereof.

The determination of which alleles is/are present in a patient suffering from chronic hepatitis C will enable the physician to establish the best hepatitis C treatment regimen for that patient (e.g., nature, dose and duration of hepatitis C treatment and/or other antiviral drugs). For example, if the above method reveals insertion alleles of the rs67272382 and/or ss469415590 polymorphism(s) are not present in a nucleic acid sample obtained from the patient, indicating that said subject is unlikely to respond to a hepatitis C treatment, then this subject can be considered as good candidate for newer treatment strategies (such as therapy with higher doses of currently available drugs, longer treatment duration with currently available drugs and/or newer drugs).

A number of methods are available for analyzing the presence or absence of at least one polymorphism, which can be applied to the IL28B region of the genome in a nucleic acid sample isolated from a biological sample obtained from a subject. Assays for detection of polymorphisms or mutations fall into several categories, including but not limited to direct sequencing assays, fragment polymorphism assays, hybridization assays, and computer based data analysis. Protocols and commercially available kits or services for performing these general methods are available. In some embodiments, assays are performed in combination or in hybrid (e.g., different reagents or technologies from several assays are combined to yield one assay). The following assays are useful in the present invention, and are described in relationship to detection of the various polymorphisms found in the IL28B region of the genome.

In one aspect of the present invention, the presence or absence of alleles is determined using a direct sequencing technique. In these assays, DNA samples are first isolated from a subject using any suitable method. In some embodiments, DNA in the region of interest is amplified using the Polymerase Chain Reaction (PCR). In other embodiments, RNA is used to generate cDNA and then perform detection analysis of the polymorphism. Following amplification, DNA or cDNA in the region of interest (e.g., the region containing the polymorphism) is sequenced using any suitable method, including but not limited to manual sequencing (e.g., using labeled marker nucleotides), or automated sequencing. The results of the sequencing are displayed using any suitable method. The sequence is examined and the presence or absence of a given allele is determined.

In one aspect of the present invention, alleles are determined using a PCR-based assay. In some embodiments, the PCR assay comprises the use of oligonucleotide primers to amplify a fragment containing the polymorphism of interest. Amplification of a target polynucleotide sequence may be carried out by any method known to the skilled artisan. See, for instance. Amplification methods include, but are not limited to, PCR including real time PCR (RT-PCR), strand displacement amplification, pyrosequencing, strand displacement amplification using Phi29 DNA polymerase (U.S. Pat. No. 5,001,050), transcription-based amplification, self-sustained sequence replication (“3SR”), the Qbeta replicase system, nucleic acid sequence-based amplification (“NASBA”), the repair chain reaction (“RCR”), boomerang DNA amplification (or “BDA”), and mismatch PCR. PCR is the preferred method of amplifying the target polynucleotide sequence.

PCR may be carried out in accordance with techniques known by the skilled artisan. In general, PCR involves, first, treating a nucleic acid sample (e.g., in the presence of a heat stable DNA polymerase) with a pair of amplification primers. One primer of the pair hybridizes to one strand of a target polynucleotide sequence. The second primer of the pair hybridizes to the other, complementary strand of the target polynucleotide sequence. The primers are hybridized to their target polynucleotide sequence strands under conditions such that an extension product of each primer is synthesized which is complementary to each nucleic acid strand. The extension product synthesized from each primer, when it is separated from its complement, can serve as a template for synthesis of the extension product of the other primer. After primer extension, the sample is treated to denaturing conditions to separate the primer extension products from their templates. These steps are cyclically repeated until the desired degree of amplification is obtained. The amplified target polynucleotide can then be used in one of the detection assays described elsewhere herein to identify the presence or absence of polymorphism of the present invention.

Because mismatches between the primer sequence and the template sequence can result in inability of the polymerase to extend the primer, and thus failure to generate an amplification product, primers designed to hybridize perfectly with one or more allele can be used to detect such alleles. While mismatches can be designed at any position on the primer, mismatches at the 3′ terminal end of the primer are most beneficial as such primers usually cannot be extended by the polymerase. For example, a primer consisting of 27 nucleotides, the first 26 of which are identical to nucleotides 1-26 of SEQ ID NO:7 the 27^{th} nucleotide being a guanidine, would successfully produce a PCR amplification product from template DNA comprising SEQ ID NO:7. However, because the 3′ guanidine would not pair with the adenine located on the opposite strand at position 28 of SEQ ID NO:1 or SEQ ID NO:5, such a primer would not produce a product from template DNA comprising SEQ ID NO:1 or SEQ ID NO:5. Thus, such a primer would be useful for discriminating between DNA comprising SEQ ID NO:1 or SEQ ID NO:5, and DNA comprising SEQ ID NO:7.

In one aspect of the present invention, polymorphisms are detected using a fragment length polymorphism assay. In a fragment length polymorphism assay, a unique DNA banding pattern based on cleaving the DNA at a series of positions is generated using an enzyme (e.g., a restriction endonuclease). DNA fragments from a sample containing a polymorphism will have a different banding pattern than wild type.

In one aspect of the present invention, polymorphism are detected by fragment sizing analysis. Such analysis can be performed using, for example, the Beckman Coulter CEQ 8000 genetic analysis system, a method well-known in the art for microsatellite polymorphism determination.

In one aspect of the present invention, the presence or absence of alleles is determined using a restriction fragment length polymorphism assay (RPLP). The region of interest is first isolated using PCR. The PCR products are then cleaved with restriction enzymes known to give a unique length fragment for a given polymorphism. The restriction-enzyme digested PCR products are separated by agarose gel electrophoresis and visualized by ethidium bromide staining, or other means know in the art, and compared to controls (wild-type).

In one aspect, polymorphisms are detected using a CLEAVASE fragment length polymorphism assay (CFLP; Third Wave Technologies, Madison, Wis.: see e.g., U.S. Pat. No. 5,888,750). This assay is based on the observation that, when single strands of DNA fold on themselves, they assume higher order structures that are highly individual to the precise sequence of the DNA molecule. These secondary structures involve partially duplexed regions of DNA such that single stranded regions are juxtaposed with double stranded DNA hairpins. The CLEAVASE I enzyme, is a structure-specific, thermostable nuclease that recognizes and cleaves the junctions between these single-stranded and double-stranded regions. Such assay is exemplified in Oldenburg, M. C., Siebert, M., “New Cleavase Fragment Length Polymorphism Method Improves the Mutation Detection Assay” 2000 Biotechniques 28,:351-357, which is incorporated herein by reference.

In other aspects of the present invention, the presence or absence of alleles detected by hybridization assay. In a hybridization assay, the presence or absence of a given allele or mutation is determined based on the ability of the DNA from the sample to hybridize to a complementary DNA molecule (e.g., an oligonucleotide probe). A variety of hybridization assays using a variety of technologies for hybridization and detection are available. A description of a selection of assays is provided below.

In a preferred aspect, the hybridized nucleic acids are detected by detecting one or more labels attached to the sample nucleic acids. The labels may be incorporated by any of a number of means well known to those of skill in the art. In one embodiment, the label is simultaneously incorporated during the amplification step in the preparation of the sample nucleic acids. For example, the polymerase chain reaction (PCR) can be performed using labeled primers or labeled nucleotides, resulting in a labeled amplification product. In another embodiment, transcription amplification using a labeled nucleotide (e.g. fluorescein-labeled UTP and/or CTP) incorporates a label into the transcribed nucleic acids.

Alternatively, a label may be added directly to the original nucleic acid sample (e.g., mRNA, polyA mRNA, cDNA, genomic DNA etc.) or to the amplification product after the amplification is completed. Means of attaching labels to nucleic acids are well known to those of skill in the art and include, for example, nick translation or end-labeling (e.g. with a labeled RNA) by kinasing the nucleic acid and subsequent attachment (ligation) of a nucleic acid linker joining the sample nucleic acid to a label (e.g., a fluorophore). In another embodiment label is added to the end of fragments using terminal deoxytransferase (TdT).

Detectable labels suitable for use in the present invention include any composition detectable by spectroscopic, photochemical, biochemical, immunochemical, electrical, optical or chemical means. Useful labels in the present invention include, but are not limited to: biotin for staining with labeled streptavidin conjugate; anti-biotin antibodies; magnetic beads (e.g., Dynabeads™); fluorescent, dyes (e.g., fluorescein, Texas Red, rhodamine, green fluorescent protein, and the like); radiolabels (e.g., H³, I¹²⁵, S³⁵, C¹⁴, or P³²); phosphorescent labels; enzymes (e.g., horse radish peroxidase, alkaline phosphatase and others commonly used in an ELISA); and calorimetric labels such as colloidal gold or colored glass or plastic (e.g., polystyrene, polypropylene, latex, etc.) beads. Appropriate labels are known to those skilled in the art.

Means of detecting such labels are well known to those of skill in the art. Thus, for example, radiolabels may be detected using photographic film or scintillation counters; fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.

The label may be added to the target nucleic acid(s) prior to, or after the hybridization. So-called “direct labels” are detectable labels that are directly attached to or incorporated into the target nucleic acid prior to hybridization. In contrast, so-called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting, labeled hybridized nucleic acids. See Tijssen, 1993, Laboratory Techniques in Biochemistry and Molecular Biology, Vol. 24; Hybridization with Nucleic Acid Probes, which is hereby incorporated by reference.

In one aspect, hybridization of a probe to the sequence of interest (e.g., polymorphism) is detected directly by visualizing a bound probe (e.g., a Northern or Southern assay; See e.g., Ausabel et al. (Eds.), 1991, Current Protocols in Molecular Biology, John Wiley & Sons, NY). In an example of such assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., agarose gel electrophoresis) and transferred to a membrane. A labeled (e.g., by incorporating a radionucleotide) probe or probes specific for the mutation being detected is allowed to contact the membrane under a condition of low, medium, or high stringency conditions. Unbound probe is removed and the presence of binding is detected by visualizing the labeled probe.

In one embodiment, polymorphisms are detected using a DNA chip hybridization assay. In this assay, a series of oligonucleotide probes are affixed to a solid support. The oligonucleotide probes are designed to be unique to a given single nucleotide polymorphism. The DNA sample of interest is contacted with the DNA “chip” and hybridization is detected. An example of such technology is a GeneChip (Affymetrix, Santa Clara, Calif.; see e.g., U.S. Pat. No. 6,045,996) assay. The GeneChip technology uses miniaturized, high-density arrays of oligonucleotide probes affixed to a “chip”. Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated from a biological sample obtained from the subject, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In another aspect, a DNA microchip containing electronically captured probes is utilized. One example of such technology is a NanoChip (Nanogen, San Diego, Calif.; see e.g., U.S. Pat. No. 6,068,818). Through the use of microelectronics, Nanogen's technology enables the active movement and concentration charged molecules to and from designated test sites on its semiconductor microchip. DNA capture probes unique to a given polymorphism or mutation are electronically placed at, or “addressed” to, specific sites on the microchip. Since DNA has a strong negative charge, it can be electronically moved to an area positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution distinct DNA probes is added until the array of specifically bound DNA probes is complete.

A test sample is then analyzed for the presence of target DNA molecules by determining which of the DNA capture probes hybridize, with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound nonspecifically bound DNA from each site, the polarity or charge of the site is reversed to negative, thereby forcing any unbound or nonspecifically bound DNA back into solution away from the capture probes. A laser-based fluorescence scanner is used to detect binding.

In yet other aspects, a “bead array” is used for the detection of polymorphisms (Illumina, San Diego, Calif.; see e.g., PCT Publications WO99/67641 and WO00/39587, each of which is herein incorporated by reference). Illumina uses a bead array technology that combines fiber optic bundles and beads that self-assemble into an array. Each fiber optic bundle contains thousands to millions of individual fibers depending on the diameter of the bundle. The beads are coated with an oligonucleotide specific for the detection of a given polymorphism or mutation. Batches of beads are combined to form a pool specific to the array. To perform an assay, the bead array is contacted with a prepared subject sample (e.g., DNA). Hybridization is detected using any suitable method, such as for example, Enzymatic Detection of Hybridization

In some aspects of the present invention, genomic profiles are generated using an assay that detects hybridization by enzymatic cleavage of specific structures. One example of such an assay is the INVADER® assay (Third Wave Technologies; see e.g., U.S. Pat. No. 6,001,567, and Olivier, M., The Invader assay for SNP Genotyping, 2005 Mutat Res. June 3; 573(1-2):103-110, both of which are incorporated herein by reference). The INVADER® assay detects specific DNA and RNA sequences by using structure-specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′-end labeled with fluorescein that is quenched by an internal dye. Upon cleavage, the dequenched fluorescein labeled product may be detected using a standard fluorescence plate reader.

In some aspects, a MassARRAY system (Sequenom, San Diego, Calif.) is used to detect polymorphisms (see e.g., U.S. Pat. No. 6,043,031).

Genomic DNA samples are usually, but need not be, amplified before being analyzed. Genomic DNA can be obtained from any biological sample. Amplification of genomic DNA containing a polymorphisms generates a single species of nucleic acid if the individual from whom the sample was obtained is homozygous at the polymorphic site, or two species of nucleic acid if the individual is heterozygous.

RNA samples also are often subject to amplification. In this case, amplification is typically, but not necessarily, proceeded by reverse transcription. Amplification of all expressed mRNA can be performed as described in, for example, in Innis M A et al., 1990. “Academic Press”. PCR Protocols: A Guide to Methods and Applications and Bustin S A 2000. “Journal of Molecular Endocrinology, 25”. Absolute quantification of mRNA using real-time reverse transcription polymerase chain reaction assays. pp. 169-193, which are hereby incorporated by reference in their entirety. Amplification of an RNA sample from a diploid sample can generate two species of target molecules if the individual providing the sample is heterozygous at a polymorphic site occurring within the expressed RNA, or possibly more if the species of the RNA is subjected to alternative splicing. Amplification generally can be performed using the polymerase chain reaction (PCR) methods known in the art. Nucleic acids in a target sample can be labeled in the course of amplification by inclusion of one or more labeled nucleotides in the amplification mixture. Labels also can be attached to amplification products after amplification (e.g., by end-labeling). The amplification product can be RNA or DNA, depending on the enzyme and substrates used in the amplification reaction.

Once a sample has been analyzed to determine which allele of a polymorphism is present, the individual can be selected, or identified, as being able to, or not being able to, spontaneously clear an HCV infection or respond to treatment for such an infection. Such a selection is made using the results from the analysis step of the disclosed method. For example, a person obtaining the result of the analysis step could then decide if the person is able to respond to a treatment for HCV infection and, if not, decide to use an alternative treatment. As a further example, a person reviewing the data obtained from the analysis step could then decide if the person is able to spontaneously clear an HCV infection and, if not, decide to begin administration of a treatment. In one embodiment, the selection is mad using a device. For example, a device could be designed such that when the insertion allele of the ss469415590 polymorphism is present, the output of the device indicates the individual is capable of spontaneously clearing an HCV infection or responding to treatment for such an infection. In one embodiment, the device is an electronic device. For example, a device that analyzes the sequence of a DNA molecule could be designed to display the result with regard to the ability of an individual to spontaneously clear an HCV infection or respond to treatment for such an infection. In one embodiment, the device comprises a microprocessor. In one embedment, the device is a computer.

Also included in the present invention are kits useful for practicing the disclosed methods of the present invention. Thus, one embodiment of the present invention is a kit for determining the likelihood of response to a hepatitis C treatment in a subject, in accordance with the present invention, said kit comprising i) reagents for selectively detecting the presence or absence of at least one polymorphism of the present invention in a nucleic acid sample isolated from a biological sample obtained from the subject and ii) instructions for using the kit.

One embodiment of the present invention is a kit for determining the likelihood of spontaneous clearance of hepatitis C virus in a subject infected with the virus, in accordance with the present invention, said kit comprising i) reagents for selectively detecting the presence or absence of at least one polymorphism of the present invention in a nucleic acid sample isolated from a biological sample obtained from the subject and ii) instructions for using the kit.

Kits of the present invention will contain at least some of the reagents required to determine the presence or absence of particular alleles of the present invention. Reagents for kits of the present invention can include, but are not limited to, an isolated nucleic acid, preferably a primer, a set of primers, or an array of primers, as described elsewhere herein. The primers may be fixed to a solid substrate. The kits may further comprise a control target nucleic acid and primers. One skilled in the art will, without undue experiments, be able to select the primers in accordance with the usual requirements. The isolated nucleic acids of the kit may also comprise a molecular label or tag. Usually, the primer, set of primers, or array of primers, are directed to detect the presence or absence of at least one allele of the present invention. In one embodiment, the kit comprises primers, or probes, for detecting at least one allele selected from the group consisting of an rs6727382 insertion allele, an rs6727382 deletion allele, an ss469415590 insertion allele and an ss469415590 deletion allele, an rs74597329 “T” allele (represented by SEQ ID NO:17), an rs74597329 “G” allele (represented by SEQ ID NO:19), an rs117648444 “C” allele (represented by SEQ ID NO:21), an rs117648444 “T” allele (represented by SEQ ID NO:23), an ss539198934 “T” allele (represented by SEQ ID NO:25) an ss539198934 “C” allele (represented by SEQ ID NO:27), an rs73555604 “G” allele (represented by SEQ ID NO:29), an rs73555604 “A” allele (represented by SEQ ID NO:31), an rs137902769 “T” allele (represented by SEQ ID NO:33), an rs137902769 “A” allele (represented by SEQ ID NO:35), an rs142981501 “G” allele (represented by SEQ ID NO:37), an rs142981501 “C” allele (represented by SEQ ID NO:39), and combinations thereof. In one embodiment, the presence or absence of at least one allele selected from the group consisting of an rs6727382 insertion allele, an rs6727382 deletion allele, an ss469415590 insertion allele and an ss469415590 deletion allele, an rs74597329 “T” allele (represented by SEQ ID NO:17), an rs74597329 “G” allele (represented by SEQ ID NO:19), an rs117648444 “C” allele (represented by SEQ ID NO:21), an rs117648444 “T” allele (represented by SEQ ID NO:23), an ss539198934 “T” allele (represented by SEQ ID NO:25) an ss539198934 “C” allele (represented by SEQ ID NO:27), an rs73555604 “G” allele (represented by SEQ ID NO:29), an rs73555604 “A” allele (represented by SEQ ID NO:31), an rs137902769 “T” allele (represented by SEQ ID NO:33), an rs137902769 “A” allele (represented by SEQ ID NO:35), an rs142981501 “G” allele (represented by SEQ ID NO:37), an rs142981501 “C” allele (represented by SEQ ID NO:39), and combinations thereof, is determined using at least one nucleic acid molecule (e.g., PCR primers, sequencing primers, probes, etc.) functionally equivalent to a polynucleotide comprising a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12, SEQ ID NO:13, SEQ ID NO:14, SEQ ID NO:17, SEQ ID NO:18, SEQ ID NO:19 SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43 and SEQ ID NO:44. As used herein, functionally equivalent means a nucleic acid molecule that slightly differs in its sequence from the polynucleotide, but that performs the same function as the polynucleotide. For example, a functionally equivalent nucleic acid molecule may be longer or shorter by 5, 10, or 15 nucleotides but still hybridize to the same site in the genome as a polynucleotide selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12, SEQ ID NO:13, SEQ ID NO:14, SEQ ID NO:17, SEQ ID NO:18, SEQ ID NO:19 SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43 and SEQ ID NO:44. A functionally equivalent nucleic acid molecule can also have several nucleotide substitutions (e.g., 5 or different nucleotides) compared to a polynucleotide selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12, SEQ ID NO:13, SEQ ID NO:14, SEQ ID NO:17, SEQ ID NO:18, SEQ ID NO:19 SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43 and SEQ ID NO:44, yet still retain the ability to determine the presence or absence of a particular allele of the present invention. Methods of designing functionally equivalent nucleic acid molecules based on the sequences disclosed herein are known to those skilled in the art. In one embodiment, the presence or absence of at least one allele selected from the group consisting of an rs6727382 insertion allele, an rs6727382 deletion allele, an ss469415590 insertion allele and an ss469415590 deletion allele is determined using at least one nucleic acid molecule (e.g., PCR primers, sequencing primers, probes, etc.) comprising a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12, SEQ ID NO:13, SEQ ID NO:14, SEQ ID NO:17, SEQ ID NO:18, SEQ ID NO:19 SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43 and SEQ ID NO:44.

In addition to the primers, set of primers, or array of primers, directed to detect the presence or absence of at least one allele of the present invention from a nucleic acid sample isolated from a biological sample obtained from a subject, the reagents of the kit may comprise, for example, another primer, set of primers, or array of primers, directed to separately detect the viral genotype isolated from a biological sample obtained from a subject. These set of primers, or array of primers used are generally known in the art or may be readily generated knowing the usual requirements.

Kits of the present invention can also comprise various reagents, such as buffers, necessary to practice the methods of the invention, as known in the art. These reagents or buffers may, for example, be useful to extract and/or purify the nucleic from the biological sample obtained from the subject. The kit may also comprise all the necessary material such as microcentrifuge tubes necessary to practice the methods of the invention.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

### EXAMPLES

### Example 1

This Example demonstrates the presence of novel polymorphisms approximately 3,500 bp upstream of the IL28B translational start site.

Primary human hepatocytes (PHH) from liver donors heterozygous for rs12979860 (C/T) and not infected with HCV were purchased from Lonza (Walkersville, Md.) or Celsis (Chicago, Ill.). Information about age, sex, BMI, cause of death and viral infection status (CMV, HCV, HIV) was available for each donor. The cells were received attached in 6-well collagen-coated plates or in suspension, within 24-48 hours of isolation from the livers. Upon receiving, cell counts and viability were evaluated for each sample. PHH shipped in suspension were centrifuged at 50 g for 5 min and plated in InVitroGRO CP medium (Celsis) in collagen-coated plates or chamber slides (BD Biosciences, San Jose, Calif.). After 6 hours, the medium was replaced with InVitroGRO HI medium supplemented with Torpedo antibiotic mix (Celsis) and incubated overnight.

Following overnight incubation, the PHH were treated with PolyI:C (Imgenex, San Diego, Calif.), which is a synthetic mimic of double-stranded HCV RNA. The PolyI:C was added directly to the cell media to a final concentration of 50 μg/ml and harvested for mRNA analysis 0, 1, 2, 4, 8 or 24 hours post-treatment.

DNase-I treated RNA was prepared from the treated PHH using an RNeasy kit (Qiagen). Total RNA (1 ug) from PHH was used for selection of PolyA mRNA transcripts and library preparation with TruSeq kit (Illumina Inc., San Diego, Calif.). Following polyA mRNA selection, the RNA samples were fragmented and ligated to 65 bp adaptors to prepare paired end (PE) cDNA libraries with fragments of 200-250 bp, according to the standard Illumina protocol. The libraries were enriched by 12 PCR cycles and sequenced at a concentration of 4.5 pM, using Genome Analyzer (GAII), generating in average 47.2±6.6 million of 107 bp paired-end sequencing reads per sample. The reference human genome was built based on UCSC hg19 index using Bowtie software. After standard quality control procedure the sequenced reads were processed using Illumina Pipeline OLB 1.9.0 and CASAVA 1.7.0 and aligned to the reference genome using TopHat v1.2.0. A library of all human transcripts from Ensemble database, version GRCh37.61: useast.ensembl.org/info/data/ftp/index was used to generate a library of exon junctions and reconstruct splicing forms. Default TopHat algorithm removes from analysis RNA-seq reads that map to more than one genomic region. Considering the complexity of the region surrounding IL28B gene, it was necessary to employ a special strategy. In particular, to allow non-exclusive mapping of reads to regions of high similarity, such as IL28A, IL28B, and IL29 and IL28A/B pseudogene, TopHat settings were changed to allow multiple alignments (up to 10). The mapping identified expression clusters, representing potential exons. Based on the created database of potential splice junctions, previously unmapped reads were re-mapped by TopHat v1.2.0. To detect novel transcripts, the final aligned read files were processed by Cufflinks v0.9.3. The TopHat alignment algorithm breaks sequence reads into 25-bp segments that are independently mapped and reconstructed back into sequence reads if all individual segments are mapped correctly. Relative abundances of transcripts were measured with fragments per kilobase of exon per million fragments mapped algorithm (FPKM). Confidence intervals for FPKM estimates were calculated using a Bayesian inference method. In the presence of splicing forms, the highest expressing form of each gene was assigned a ratio 1, and all other forms expressed at least at 1% level of the main form (ratio>0.01) were used for analysis. To discover possible genetic variants, 2 or 3 mismatches per each 25-bp segment were allowed. Mapping results were visualized using both the UCSC genome browser and a local copy of the Integrative Genomics Viewer (IGV) software: broadinstitute.org/igv/. Genetic variants in the IL28B/IL29 region were visualized by IGV and then examined manually and validated by Sanger sequencing in DNA and cDNA samples.

An analysis of RNA-seq data that focused on a 150-Kb region around rs12979860 showed concordance with induction of the IFN-2 genes (IL28A, IL28B and IL29) genes, as determined by TaqMan expression analysis. Expression of IFN-2 genes was not observed in the absence of PolyI:C treatment, but was observed after 2-24 hours of PolyI:C activation (FIG. 1a).

Transient activation of a novel transcribed region was also observed upstream of IL28B, with the highest levels of expression detected at 2 and 4 hours (FIG. 1b). Analysis of paired-end RNA-seq reads identified one major splice junction site. This common sequence was then used as a starting point for 5′ rapid amplification of cDNA ends (5′RACE). Briefly, RACE was performed with SMARTer RACE cDNA kit (Clontech, Mountain View, Calif.) using the DNAse-I treated RNA samples from PolyI:C-treated PHH. The transcripts were PCR-amplified with specific cloning primers (Supplementary Table 11) from cDNA of PolyI:C-treated PHHs. PCR reactions were performed with AmpliTaq Gold 360 Master Mix (Life Technologies) and 360 GC Enhancer (Life Technologies) using the touchdown PCR program with initial denaturation step with 10 minutes at 95° C., followed by 20 cycles (30 seconds at 95° C., 45 seconds for 2 cycles at each temperature from 70 through 60° C. decreasing by 1° C. at each step, 45 seconds at 72° C.); 25 additional cycles (30 seconds at 95° C., 45 seconds at 60° C. and 45 seconds at 72° C.); and final extension time of 7 minutes at 72° C. Gel-purified PCR fragments were cloned into a C-terminal pFC14A-Halo tag expression vector (Promega) and sequenced for validation. This analysis identified a transcription start site as well as a novel compound dinucleotide variant, denoted ss469415590 TT/ΔG, comprised of a one-base insertion/deletion (indel) polymorphism (T/A, rs67272382) and a one-base substitution variant (T/G, rs74597329). Using PolyI:C-stimulated PHH from 5 additional liver donors, ten individual transcripts created by a combination of the ss469415590 alleles were cloned and annotated. The location of these novel transcripts 3 Kb upstream of and in the same orientation as IL28B, raised the possibility that they are alternative splicing forms of IL28B or fusions. However, the presence of a CTCF transcriptional insulator site (Phillips, J. E. & Corces, V. G. CTCF: master weaver of the genome. Cell 137, 1194-211 (2009); Rosenbloom, K. R. et al. ENCODE whole-genome data in the UCSC Genome Browser: update 2012. Nucleic Acids Res 40, D912-7 (2012)) between the two transcribed regions (FIG. 1b), the results of the RACE experiments and the failure to generate an RT-PCR product between IL28B and the novel transcribed region, confirmed their independence. Despite high overall similarity with a genomic region upstream of IL28A, the novel transcripts and ss469415590 are specific for the region upstream of IL28B (FIG. 2).

### Example 2

This Example demonstrates the frequency of rs12979860 and ss469415590 alleles in various populations.

The GWAS markers rs12979860 and rs8099917 are located 367 bp downstream—and 4 kb upstream of ss469415590, respectively. Analysis of the HapMap (The International HapMap Project. Nature 426, 789-96 (2003)) (FIG. 5) and the 1000 Genomes project (Consortium, G. P. A map of human genome variation from population-scale sequencing. Nature 467, 1061-73 (2010)) data (FIG. 6) showed that the ss469415590-AG allele is perfectly correlated with the unfavorable rs12979860-T allele in Asians (CHB+JPT sets, r²=1.00) and well correlated in Europeans (CEU set, HapMap, r²=0.92; 1000 Genomes, r²=0.83). In Africans, however, this correlation is only moderate (YRI set, HapMap, r²=0.71; 1000 Genomes, r²=0.65), even though rs12979860 is the best surrogate for ss469415590 of all markers within the 100-Kb region. Correlation between ss469415590 and rs8099917 is high in Asians (r²=0.91), moderate in Europeans (r²=0.44), but very low in Africans (r²=0.008) (FIG. 5).

### Example 3

This Example demonstrates association of rs12979860 and ss469415590 with spontaneous clearance of hepatitis C virus infection.

The association of ss469415590 and rs12979860 with HCV clearance was assessed in 1,436 African-American and 1,480 European-American individuals from four studies:

### 1. VirahepC Study

The Study of Viral Resistance to Antiviral Therapy of Chronic Hepatitis C (Virahep-C) was designed to compare response to treatment with pegylated IFN-a/ribavirin in African American patients with chronic hepatitis C to otherwise similar patients of European ancestry (Conjeevaram, H. S. et al., 2006 Peginterferon and ribavirin treatment in African American and Caucasian American patients with hepatitis C genotype 1. Gastroenterology 131, 470-7). In Virahep-C, patients with HCV genotype 1 infection who had not undergone previous treatment for chronic hepatitis C received treatment with a standard regimen of pegylated IFN-alfa-2a (180 μg/week) plus ribavirin (1000-1200 mg/day) for up to 48 weeks. Ancestral designation was self-reported. Study end points included: decrease in HCV RNA levels between baseline and various treatment time points; week 24 response (absence of detectable HCV RNA in serum after 24 weeks of therapy); end-of-treatment response (absence of HCV RNA after 48 weeks of therapy); and sustained virologic response (SVR; absence of HCV RNA 24 weeks after treatment was stopped). The protocol was approved by the institutional review boards of the participating institutions and all patients gave informed written consent. Reported results from Virahep-C showed that African-American patients had lower rates of virologic response than European-American patients and that those differences were not explained by differences in patient characteristics, baseline HCV RNA levels or the amount of medication taken during the study (Conjeevaram, H. S. et al., 2006 Peginterferon and ribavirin treatment in African American and Caucasian American patients with hepatitis C genotype 1. Gastroenterology 131, 470-7).

### 2. HALT-C Study

The Hepatitis C Antiviral Long-term Treatment against Cirrhosis (HALT-C) Trial was a study of patients with advanced chronic hepatitis C who had failed previous interferon-based treatment (DiBisceglie, A. M. et al. 2008 Prolonged therapy of advanced chronic hepatitis C with low-dose peginterferon. N Engl J Med 359, 2429-41; Lee, W. M. et al. 2004 Evolution of the HALT-C Trial: pegylated interferon as maintenance therapy for chronic hepatitis C in previous interferon nonresponders. Control Clin Trials 25, 472-92). At enrollment, HALT-C patients had an Ishak fibrosis score >3 by local assessment of liver biopsy, had a Child-Turcotte-Pugh score <7 and had no evidence of hepatocellular carcinoma. Final assessment of fibrosis stage was performed by a panel of hepatopathologists. Patients with other liver diseases, human immunodeficiency virus infection, active illicit drug use or current alcohol abuse were excluded. Ancestral designation was self-reported. During the lead-in phase of HALT-C, patients underwent retreatment with pegylated-interferon-alfa-2a (180 μg/week) plus ribavirin (1000-1200 mg/day). Subjects with undetectable HCV RNA at week 20 remained on combination treatment through week 48 and were followed until week 72. Subjects with undetectable HCV RNA at weeks 48 and 72 were considered to have an SVR. Investigations of human genetics in the HALT-C Trial were conducted in those participants who provided (written) consent for genetic testing. The HALT-C Trial was approved by institutional review boards of the participating institutions.

### 3. Urban Health Study (UHS) Study

As previously described (Shebel, F. M., et al. IL28B rs12979860 Genotype and Spontaneous Clearnace of Hepatitis C Virus in Multi-Ethnic Cohort of Injection Drug Users: Evidence for a Supra-Additive Association. J Infect Dis 204, 1843-7), UHS recruited IDUs from street settings in six inner-city San Francisco Bay area neighborhoods from 1986 through 2002, drawing serial cross-sectional samples every six months (Watters, J. K. et al 1995, HIV seroprevalence in in jection drug users. JAMA 273, 1178).

Individuals 18 years of age or older were eligible for enrollment if they had injected drugs within the past 30 days or previously enrolled in the UHS study. New participants were screened for visible signs of recent or chronic injection (i.e., venipuncture sites or scars). Interviews were conducted using standardized questionnaires and blood samples were collected from participants who provided written informed consent. The present study included unduplicated IDUs recruited between 1998 and 2000 (Tseng, F. C. et al. 2007, Seroprevalence of hepatitis C virus and hepatitis B virus among San Francisco injection drug users, 1998 to 2000. Hepatology 46, 666-71) Participants who were positive for HCV antibody were divided into two groups based on their HCV RNA result: ‘chronic’ (positive for HCV RNA) or ‘cleared’ (negative for HCV RNA and positive for antibody). All subjects with cleared infection were included in the study and frequency matched to those with chronic infection (maximum 4:1) on the basis of self-reported ethnicity and age.

Information on demographic variables and other potential covariates were assessed through face-to-face personal interviews (Tseng, F. C. et al. 2007, Seroprevalence of hepatitis C virus and hepatitis B virus among San Francisco injection drug users, 1998 to 2000. Hepatology 46, 666-71; Kral, A. H., et al. 2001, Sexual transmission of HIV-1 among injection drug users in San Francisco, USA: risk-factor analysis. Lancet 357, 1397-401). After being interviewed participants were counseled by trained staff on reducing infection risks and referred to appropriate medical and social services. Participants were not asked about treatment for HCV infection during 1998-2000, but when they were asked during 2001-2002, reports of antiviral treatment were rare (Seal, K. H. et al. 2005, Among injection drug users, interest is high, but access low to HCV antiviral therapy. Society of Generl Internal Medicine, 28^{th} annual meeting, New Orleans, La., USA, May 11-24. J Gen Intern Med 20, Suppl 1, 171); thus it is very likely that the HCV seropositive, HCV RNA negative subjects in this study had recovered spontaneously. For the purpose of genetic investigations, ancestry was ascertained by self report; subjects who reported themselves to be White and not of Latino/Hispanic ethnicity are considered to be of ‘European American’ ancestry. Study procedures were approved by an Institutional Review Board of the National Cancer Institute and the Committee on Human Subjects Research at the University of California, San Francisco.

### 4. ALIVE Study

The AIDS Link to Intravenous experience (ALIVE) is an ongoing study of injection drug users enrolled in Baltimore, Md., from February 1988 through March 1989 (Vlahov, D. et al. 1991, The ALIVE study, a longitudinal study of HIV-1 infection in intravenous drug users: descriptions of methods and characteristics of participants. NIDA Res Monogr 109, 75-100). HCV infection was established by detection of HCV antibody (anti-HCV) by enzyme immunoassay (EIA) and recombinant immunoblot assay (RIBA [version 3.0]; Novartis). Individuals with cleared HCV infection had anti-HCV (as confirmed by RIBA) and undetectable HCV RNA in serum or plasma without having received any HCV therapy. Individuals with persistent infection had anti-HCV and HCV RNA in serum or plasma before receiving any HCV therapy. Written informed consent for genetic testing was obtained from all participants. The study was approved by the institutional review board at Johns Hopkins University.

All statistical comparisons of ss469415590 and rs12979860 were limited to subjects whose DNA specimen was successfully genotyped for both variants at the Laboratory of Translational Genomics, National Cancer Institute, blindly to clinical phenotypes.

The Kruskal-Wallis test was used to compare median HCV RNA levels between genotypes for each variant (e.g., ss469415590-TT/TT versus ss469415590-AG/AG). The mean HCV RNA levels in each of the three ss469415590 genotype groups (i.e., AG/AG, AG/TT, TT/TT) were compared with the respective rs12979860 genotype groups (TT, CT, CC). To determine global statistical significance of these three mean differences, the covariance matrix of the mean differences was computed using a bootstrap procedure. Individuals in the study were re-sampled with replacement, and the three differences of the mean RNA levels in the three genotype groups in this bootstrap dataset computed. This calculation was repeated 1000 times, and the bootstrap replicates used to compute the covariance matrix of the mean differences of the original sample. This covariance matrix was used to compute a three degrees-of-freedom Wald statistic to test the null hypothesis that there was no difference in mean HCV RNA decreases for ss469415590 and rs12979860.

For dichotomous outcomes (e.g., sustained virological response, spontaneous clearance) the odds ratio was calculated and accompanying p-value was calculated using proc logistic in SAS 9.2 TS2M3; p-values are Wald chi-square estimates. The area under the receiver operating characteristic curve (AUC) (Pepe, M. S. 2003, The Statistical Evaluation of Medical tests for Classification and Prediction, (Oxford University Press, Oxford) was also determined. To test for differences in AUC values, a p-value was calculated based on a chi-square test (1 df) that used a bootstrap variance estimate computed by resampling subjects with SVR and non-responders with replacement and then repeating the AUC computations for each bootstrap sample.

In VirahepC (Conjeevaram, H. S. et al. Peginterferon and ribavirin treatment in African American and Caucasian American patients with hepatitis C genotype 1. Gastroenterology 131, 470-7 (2006)) and HALT-C (Di Bisceglie, A. M. et al. Prolonged therapy of advanced chronic hepatitis C with low-dose peginterferon. N Engl J Med 359, 2429-41 (2008)), the response to pegIFN-α/RBV therapy in patients with CHC was evaluated (FIG. 7). The rates of sustained virological response (SVR) among the subjects from these studies, which reflect well known racial differences in response to treatment and the differing selection criteria for these clinical trials, were: Virahep C European-American, 52%; Virahep C African-American, 28%; HALT-C European-American, 18%; African-American, 7%. Spontaneous HCV clearance was also evaluated in injection drug users enrolled in two studies, UHS (Shebl, F. M. et al. IL28B rs12979860 genotype and spontaneous clearance of hepatitis C virus in a multi-ethnic cohort of injection drug users: evidence for a supra-additive association. J Infect Dis 204, 1843-7 (2011)) and ALIVE (Vlahov, D. et al. The ALIVE study, a longitudinal study of HIV-1 infection in intravenous drug users: description of methods and characteristics of participants. NIDA Res Monogr 109, 75-100 (1991)) (FIG. 8). As part of this study, the decrease in HCV RNA during the first 28 days of treatment of African-American Virahep-C participants was evaluated. This is a powerful early predictor of ultimate treatment response (FIG. 9). The decline in HCV RNA levels was more strongly associated with ss469415590 genotype than with rs12979860 genotype (p=0.015, difference in mean values; FIG. 10). In the same study, a stronger association for ss469415590 than for rs12979860 with other measures of treatment response (week 24 response, end-of-treatment response and SVR was observed; (FIG. 11), although these differences did not reach statistical significance. The association pattern was similar in African-American patients from the HALT-C study, with a stronger association for ss469415590 than for rs12979860 (week 20 response, end-of-treatment and SVR, (FIG. 12). Spontaneous HCV clearance among African-Americans was evaluated using the area under the receiver operating curve (AUC). In UHS participants, the AUC value was greater for ss469415590 (0.62) than rs12979860 (0.58) (FIG. 13). In the ALIVE study, which has higher percentage of HCV/HIV co-infected patients, the AUC values were similar for rs12979860 (0.64) and ss469415590 (0.64; (FIG. 14).

Virahep-C, HALT-C and UHS also enrolled European-American participants. In these subjects, ss469415590 and rs12979860 showed similar associations for both treatment-induced and spontaneous HCV clearance (FIGS. 10, 11, 12 and 13). Taken as a whole, the results show that among African-American individuals, ss469415590 is a better marker than rs12979860 for predicting response to pegIFN-α/RBV treatment of CHC and possibly for spontaneous HCV clearance, while these variants are similarly informative in European-Americans.

### Example 4

This Example demonstrates the identification of additional polymorphisms surrounding ss469415590 TT/AG.

Further analysis of the region surrounding the ss469415590 polymorphism in 270 HapMap samples from three ethnic groups (Europeans, Africans and Asians) revealed the presence of additional variants. The allele frequencies for these variants and linkage disequilibrium with other markers are presented in FIGS. 15-18. (FIGS. 15-18). Based on a haplotype analysis of 16 markers from the 8-Kb IL28B upstream region, eight markers that captured all haplotypes present in HapMap sets were identified (FIG. 18). These eight markers were also tested in European-American and African-American patients from Virahep-C. In all populations, the unique favorable haplotype included the ss469415590-TT allele. The unfavorable ss469415590-AG allele was found on a number of haplotypes, including two haplotypes that were reported as being neutral in Europeans despite carrying the unfavorable rs12979860-T allele (Smith, K. R. et al. Identification of improved IL28B SNPs and haplotypes for prediction of drug response in treatment of hepatitis C using massively parallel sequencing in a cross-sectional European cohort. Genome Med 3, 57 (2011); Fischer, J. et al. Combined effects of different interleukin-28B gene variants on the outcome of dual combination therapy in chronic hepatitis C virus type 1 infection. Hepatology 55, 1700-10 (2012)); these two haplotypes include either the minor allele of variant rs73555604 or the minor allele of variant rs117648444. It is possible, therefore, that these variants modify the risk in carriers of the unfavorable ss469415590-AG allele and are the source of haplotype heterogeneity previously reported in Europeans (Smith, K. R. et al. Identification of improved IL28B SNPs and haplotypes for prediction of drug response in treatment of hepatitis C using massively parallel sequencing in a cross-sectional European cohort. Genome Med 3, 57 (2011); Fischer, J. et al. Combined effects of different interleukin-28B gene variants on the outcome of dual combination therapy in chronic hepatitis C virus type 1 infection. Hepatology 55, 1700-10 (2012)) (FIG. 18). In Africans, in addition to the same markers rs73555604 and rs117648444, two other variants might be of possible importance for further clarification of risk—ss539198934 and rs142981501. This suggests that a combination of ss469415590-ΔG and some or all of these markers—ss539198934, rs137902769, rs117648444, rs142981501 and rs73555604, might provide best prediction for HCV clearance.

The breadth and scope of the present invention should not be limited by any of the above-described exemplary embodiments, but should be defined only in accordance with the following claims and their equivalents.

1-27. (canceled)

28. A method for predicting the ability of an individual to spontaneously clear an HCV infection, the method comprising:
a) obtaining a biological sample selected from the group consisting of blood, saliva, urine, a skin scraping, a tissue sample and a buccal swab, from the individual;
b) analyzing the sample to identify which alleles of the ss469415590 polymorphism are present in the individual;
c) selecting the individual as being able to spontaneously clear the HCV infection if the ss469415590 insertion allele is present in the individual; or,
d) selecting the individual as being unable to spontaneously clear the HCV infection if the ss469415590 deletion allele is present in the individual.

29. The method of claim 28, wherein the individual is infected with HCV.

30. The method of claim 28, wherein the individual is not infected with HCV.

31. The method of claim 28, wherein the likelihood of the individual spontaneously clearing an HCV infection has an odds ratio of 1.2.

32. The method of claim 28, wherein the analyzing step comprises extracting DNA from the biological sample.

33. The method of claim 28, wherein identification of which ss469415590 allele is present comprises determining the identity of the nucleotide present in position 27 of SEQ ID NO:1 and/or position 28 of SEQ ID NO:1.

34. The method of claim 33, wherein identification of the nucleotide present in position 27 of SEQ ID NO:1 and/or position 28 of SEQ ID NO:1 comprises conducting PCR analysis using at least one primer selected from the group consisting of:
a) a primer that specifically hybridizes to a nucleic acid molecule comprising SEQ ID NO:6 at a location immediately downstream of the nucleotide corresponding to position 26 of SEQ ID NO:6, wherein the 3′ terminal nucleotide of the primer is thymidine;
b) a primer that specifically hybridizes to a nucleic acid molecule comprising SEQ ID NO:5 at a location immediately downstream of the nucleotide corresponding to position 28 of SEQ ID NO:5, wherein the 3′ terminal nucleotide of the primer is adenine;
c) a primer that specifically hybridizes to a nucleic acid molecule comprising SEQ ID NO:8 at a location immediately downstream of the nucleotide corresponding to position 25 of SEQ ID NO:8, wherein the 3′ terminal nucleotide of the primer is guanine;
d) a primer that specifically hybridizes to a nucleic acid molecule comprising SEQ ID NO:7 at a location immediately downstream of the nucleotide corresponding to position 27 of SEQ ID NO:7, wherein the 3′ terminal nucleotide of the primer is cytosine;
e) a primer that specifically hybridizes to a nucleic acid molecule comprising SEQ ID NO:6 at a location comprising nucleotides 25 and 26 of SEQ ID NO:6, wherein the primer comprises a nucleotide sequence 100% identical to at least nucleotides 26-29 of SEQ ID NO:5;
f) a primer that specifically hybridizes to a nucleic acid molecule comprising SEQ ID NO:8 at a location comprising nucleotides 25 and 26 of SEQ ID NO:8, wherein the primer comprises a nucleotide sequence 100% identical to at least nucleotides 25-28 of SEQ ID NO:7; and,
g) a primer fully complementary to a primer of e) or f).

35. A method for predicting the clinical response of an individual suffering from an HCV infection to administration of a therapeutic treatment, the method comprising:
a) obtaining a biological sample selected from the group consisting of blood, saliva, urine, a skin scraping, a tissue sample and a buccal swab, from the individual;
b) analyzing the sample to identify which alleles of the ss469415590 polymorphism are present in the individual;
c) selecting the individual as predicted to respond to the administration of the therapeutic treatment if the ss469415590 insertion allele is present in the individual; or,
d) selecting the individual as predicted to respond to the administration of the therapeutic treatment if the ss469415590 deletion allele is present in the individual.

36. The method of claim 35, wherein the likelihood of the individual responding to treatment has an odds ratio of 1.2.

37. The method of claim 35, wherein the treatment comprises the administration of at least one of an interferon, ribavirin and a direct acting antiviral agent to the individual.

38. The method of claim 35, wherein identification of which ss469415590 allele is present comprises determining the identity of the nucleotide present in position 27 of SEQ ID NO:1 and/or position 28 of SEQ ID NO:1.

39. The method of claim 38, wherein identification of the nucleotide present in position 27 of SEQ ID NO:1 and/or position 28 of SEQ ID NO:1 comprises conducting PCR analysis using at least one primer selected from the group consisting of:
a) a primer that specifically hybridizes to a nucleic acid molecule comprising SEQ ID NO:6 at a location immediately downstream of the nucleotide corresponding to position 26 of SEQ ID NO:6, wherein the 3′ terminal nucleotide of the primer is thymidine;
b) a primer that specifically hybridizes to a nucleic acid molecule comprising SEQ ID NO:5 at a location immediately downstream of the nucleotide corresponding to position 28 of SEQ ID NO:5, wherein the 3′ terminal nucleotide of the primer is adenine;
c) a primer that specifically hybridizes to a nucleic acid molecule comprising SEQ ID NO:8 at a location immediately downstream of the nucleotide corresponding to position 25 of SEQ ID NO:8, wherein the 3′ terminal nucleotide of the primer is guanine;
d) a primer that specifically hybridizes to a nucleic acid molecule comprising SEQ ID NO:7 at a location immediately downstream of the nucleotide corresponding to position 27 of SEQ ID NO:7, wherein the 3′ terminal nucleotide of the primer is cytosine;
e) a primer that specifically hybridizes to a nucleic acid molecule comprising SEQ ID NO:6 at a location comprising nucleotides 25 and 26 of SEQ ID NO:6, wherein the primer comprises a nucleotide sequence 100% identical to at least nucleotides 26-29 of SEQ ID NO:5;
f) a primer that specifically hybridizes to a nucleic acid molecule comprising SEQ ID NO:8 at a location comprising nucleotides 25 and 26 of SEQ ID NO:8, wherein the primer comprises a nucleotide sequence 100% identical to at least nucleotides 25-28 of SEQ ID NO:7; and,
g) a primer fully complementary to a primer of e) or f).

40. A method of treating a HCV infection in a patient comprising:
a) obtaining a biological sample selected from the group consisting of blood, saliva, urine, a skin scraping, a tissue sample and a buccal swab, from the patient;
b) analyzing the sample to identify which alleles of the ss469415590 polymorphism are present in the patient;
c) withholding administration of a therapeutic dose of at least one of ribavirin, an interferon, and an antiviral drug to the patient if the ss469415590 insertion allele is present in the patient; or,
d) administering a therapeutic dose of at least one of ribavirin, an interferon, and an antiviral drug to the patient if the ss469415590 deletion allele is present in the patient.

41. The method of claim 40, wherein the patient administered at least one of ribavirin, an interferon, and an antiviral drug is further administered a drug selected from the group consisting of an anti-protease drug, a second antiviral drug, and combinations thereof.

42. The method of claim 40, wherein identification of which ss469415590 allele is present comprises determining the identity of the nucleotide present in position 27 of SEQ ID NO:1 and/or position 28 of SEQ ID NO:1.

43. The method of claim 42, wherein identification of the nucleotide present in position 27 of SEQ ID NO:1 and/or position 28 of SEQ ID NO:1 comprises conducting PCR analysis using at least one primer selected from the group consisting of:
a) a primer that specifically hybridizes to a nucleic acid molecule comprising SEQ ID NO:6 at a location immediately downstream of the nucleotide corresponding to position 26 of SEQ ID NO:6, wherein the 3′ terminal nucleotide of the primer is thymidine;
b) a primer that specifically hybridizes to a nucleic acid molecule comprising SEQ ID NO:5 at a location immediately downstream of the nucleotide corresponding to position 28 of SEQ ID NO:5, wherein the 3′ terminal nucleotide of the primer is adenine;
c) a primer that specifically hybridizes to a nucleic acid molecule comprising SEQ ID NO:8 at a location immediately downstream of the nucleotide corresponding to position 25 of SEQ ID NO:8, wherein the 3′ terminal nucleotide of the primer is guanine;
d) a primer that specifically hybridizes to a nucleic acid molecule comprising SEQ ID NO:7 at a location immediately downstream of the nucleotide corresponding to position 27 of SEQ ID NO:7, wherein the 3′ terminal nucleotide of the primer is cytosine;
e) a primer that specifically hybridizes to a nucleic acid molecule comprising SEQ ID NO:6 at a location comprising nucleotides 25 and 26 of SEQ ID NO:6, wherein the primer comprises a nucleotide sequence 100% identical to at least nucleotides 26-29 of SEQ ID NO:5;
f) a primer that specifically hybridizes to a nucleic acid molecule comprising SEQ ID NO:8 at a location comprising nucleotides 25 and 26 of SEQ ID NO:8, wherein the primer comprises a nucleotide sequence 100% identical to at least nucleotides 25-28 of SEQ ID NO:7; and,
g) a primer fully complementary to a primer of e) or f).


SEQUENCE LISTING (44 sequences)

SEQ ID NO: 1 - DNA, 52 bp, Homo sapiens
gcgagagcaggcagcgccggggggccttctgcgatcaccgtgcacaggaccc

SEQ ID NO: 2 - DNA, 52 bp, Homo sapiens
gggtcctgtgcacggtgatcgcagaaggccccccggcgctgcctgctctcgc

SEQ ID NO: 3 - DNA, 51 bp, Homo sapiens
gcgagagcaggcagcgccggggggcctctgcgatcaccgtgcacaggaccc

SEQ ID NO: 4 - DNA, 51 bp, Homo sapiens
gggtcctgtgcacggtgatcgcagaggccccccggcgctgcctgctctcgc

SEQ ID NO: 5 - DNA, 52 bp, Homo sapiens; identical to SEQ ID NO: 1

SEQ ID NO: 6 - DNA, 52 bp, Homo sapiens; identical to SEQ ID NO: 2

SEQ ID NO: 7 - DNA, 51 bp, Homo sapiens
gcgagagcaggcagcgccggggggccgctgcgatcaccgtgcacaggaccc

SEQ ID NO: 8 - DNA, 51 bp, Homo sapiens
gggtcctgtgcacggtgatcgcagcggccccccggcgctgcctgctctcgc

SEQ ID NO: 9 - DNA, 21 bp, Artificial Sequence
  Synthetic
gcctgctgcagaagcagagat

SEQ ID NO: 10 - DNA, 19 bp, Artificial Sequence
  Synthetic
gctccagcgagcggtagtg

SEQ ID NO: 11 - DNA, 13 bp, Artificial Sequence
  Synthetic
atcgcagaaggcc

SEQ ID NO: 12 - DNA, 13 bp, Artificial Sequence
  Synthetic
atcgcagcggccc

SEQ ID NO: 13 - DNA, 19 bp, Artificial Sequence
  Synthetic
cgaaccagggttgaattgc

SEQ ID NO: 14 - DNA, 20 bp, Artificial Sequence
  Synthetic
gcactgcagacaggagtgag

SEQ ID NO: 15 - DNA, 135 bp, Homo sapiens
  misc_feature (52)..(52): n is a, c, g, or t
gcctgctgcagaagcagagatgcggccgagtgtctgggccgcagtggccgcngggctgtgggtcctgtncacggtgatcgcagnnggccccccggcgctgcctgctctcncactaccgctcgctggagccccgga

SEQ ID NO: 16 - DNA, 135 bp, Homo sapiens
  misc_feature (26)..(26): n is a, c, g, or t
tccggggctccagcgagcggtagtgngagagcaggcagcgccggggggccnnctgcgatcaccgtgnacaggacccacagcccngcggccactgcggcccagacactcggccgcatctctgcttctgcagcaggc

SEQ ID NO: 17 - DNA, 52 bp, Homo sapiens
cgagagcaggcagcgccggggggccttctgcgatcaccgtgcacaggaccca

SEQ ID NO: 18 - DNA, 52 bp, Homo sapiens
tgggtcctgtgcacggtgatcgcagaaggccccccggcgctgcctgctctcg

SEQ ID NO: 19 - DNA, 52 bp, Homo sapiens
cgagagcaggcagcgccggggggcctgctgcgatcaccgtgcacaggaccca

SEQ ID NO: 20 - DNA, 52 bp, Homo sapiens
tgggtcctgtgcacggtgatcgcagcaggccccccggcgctgcctgctctcg

SEQ ID NO: 21 - DNA, 53 bp, Homo sapiens
caactgctccttccgccccaggagggatcctccgcggccatccgtgaggcccg

SEQ ID NO: 22 - DNA, 53 bp, Homo sapiens
cgggcctcacggatggccgcggaggatccctcctggggcggaaggagcagttg

SEQ ID NO: 23 - DNA, 53 bp, Homo sapiens
caactgctccttccgccccaggagggattctccgcggccatccgtgaggcccg

SEQ ID NO: 24 - DNA, 53 bp, Homo sapiens
cgggcctcacggatggccgcggagaatccctcctggggcggaaggagcagttg

SEQ ID NO: 25 - DNA, 79 bp, Homo sapiens
cactctgggccttgcctctgaccccgccccctctggcagcacggaaacctccacgccattggctgccgaaagcagctcc

SEQ ID NO: 26 - DNA, 79 bp, Homo sapiens; identical to SEQ ID NO: 25

SEQ ID NO: 27 - DNA, 79 bp, Homo sapiens
ggagctgctttcggcagccaatggcgtggaggtttccgtgctgccagagggggcggggtcagaggcaaggcccagagtg

SEQ ID NO: 28 - DNA, 79 bp, Homo sapiens
ggagctgctttcggcagccaatggcgtggaggtttccgtgctgccggagggggcggggtcagaggcaaggcccagagtg

SEQ ID NO: 29 - DNA, 32 bp, Homo sapiens
gggctgtgggtcctgtgcacggtgatcgcagc

SEQ ID NO: 30 - DNA, 32 bp, Homo sapiens
gctgcgatcaccgtgcacaggacccacagccc

SEQ ID NO: 31 - DNA, 32 bp, Homo sapiens
gggctgtgggtcctgtacacggtgatcgcagc

SEQ ID NO: 32 - DNA, 32 bp, Homo sapiens
gctgcgatcaccgtgtacaggacccacagccc

SEQ ID NO: 33 - DNA, 30 bp, Homo sapiens
ctggctgcacactctgggccttgcctctga

SEQ ID NO: 34 - DNA, 30 bp, Homo sapiens
tcagaggcaaggcccagagtgtgcagccag

SEQ ID NO: 35 - DNA, 33 bp, Homo sapiens
cggctggctgcacactcagggccttgcctctga

SEQ ID NO: 36 - DNA, 33 bp, Homo sapiens
tcagaggcaaggccctgagtgtgcagccagccg

SEQ ID NO: 37 - DNA, 57 bp, Homo sapiens
gaggaagaggcgctgagctgggggcagcgcaactgctccttccgccccaggagggat

SEQ ID NO: 38 - DNA, 57 bp, Homo sapiens
atccctcctggggcggaaggagcagttgcgctgcccccagctcagcgcctcttcctc

SEQ ID NO: 39 - DNA, 57 bp, Homo sapiens
gaggaagaggcgctgagctgggggcagcccaactgctccttccgccccaggagggat

SEQ ID NO: 40 - DNA, 57 bp, Homo sapiens
atccctcctggggcggaaggagcagttgggctgcccccagctcagcgcctcttcctc

SEQ ID NO: 41 - DNA, 20 bp, Artificial Sequence
  Synthetic
gcctgtcgtgtactgaacca

SEQ ID NO: 42 - DNA, 19 bp, Artificial Sequence
  Synthetic
gcgcggagtgcaattcaac

SEQ ID NO: 43 - DNA, 15 bp, Artificial Sequence
  Synthetic
ctggttcacgccttc

SEQ ID NO: 44 - DNA, 14 bp, Artificial Sequence
  Synthetic
tggttcgcgccttc